(19) United States
(12) Patent Application Publication
(10) Pub. No.: US 2024/0368232 A1
(43) Pub. Date: Nov. 7, 2024

(54) ANTIVIRAL LARAZOTIDE DERIVATIVES

(71) Applicants: Alessio FASANO, Boston, MA (US); Simone DI MICCO, Salerno (IT)

(72) Inventors: Alessio FASANO, Boston, MA (US); Simone DI MICCO, Salerno (IT)

(21) Appl. No.: 18/568,196

(22) PCT Filed: Apr. 12, 2022
(86) PCT No.: PCT/IB2022/053413

§ 371 Date: Dec. 7, 2023

(30) Foreign Application Priority Data
Jun. 8, 2021 (IT) 102021000014879

(51) Int. Cl. C07K 14/28; A61K 38/00; A61P 31/12; C07K 5/103; C07K 5/113; C07K 5/117

(52) U.S. Cl. CPC C07K 14/28; A61P 31/12; C07K 5/101; C07K 5/1021; C07K 5/1024; A61K 38/00

(57) ABSTRACT

The present invention relates to new synthetic molecules with antiviral activity, pharmaceutical compositions comprising them and uses thereof, in particular for the prevention and/or treatment of viral infections such as SARS-COV-2 infections.

### TECHNICAL FIELD

The present invention relates to new synthetic molecules with antiviral activity, pharmaceutical compositions comprising them and uses thereof, in particular for the prevention and/or treatment of viral infections such as SARS-COV-2 infections.

### Background

Viral infections, and particularly infections of the respiratory tract, represent a major public health problem because of their worldwide occurrence and considerable morbidity and mortality. Notably, COVID-19 is a serious and potentially life-threatening disease triggered by the SARS-COV-2 virus. To date, the COVID-19 pandemic has infected ˜73 million people around the world and claimed nearly 1.7 million lives.

The most severe outcome of COVID-19 infection is the development of interstitial pneumonia causing acute lung injury (ALI) and/or acute respiratory distress syndrome (ARDS), both responsible for the infected patients' mortality. ALI and ARDS are characterized by a leakage of plasma components into the lungs, compromising their ability to expand and optimally engage in gas exchange with blood, resulting in respiratory failure. Available treatment for ALI/ARDS in this patient population is limited and there is currently no proven and effective cure that has been approved for treatment of SARS-COV-2 infection.

Within this context, there is hence an urgent need for identifying novel therapeutic targets and developing safe and effective therapeutic options for the prevention and/or treatment of viral infections.

Recently, Yang et al. reported the first X-ray structure of the main protease of the COVID-19 virus (M^{pro}, PDB ID: 6L U7) (Jin et al., 2020) covalently bound to N3 (FIG. 1), a peptide-like inhibitor acting in low micromolar range. Another research group has independently resolved the M^{pro} crystallographic structure covalently linked to another inhibitor characterized by an α-ketoamide structure (Zhang et al., 2020), with crystallographic resolution similar to the N3-M^{pro} complex described above.

The M^{pro} enzyme is instrumental for viral transcription and replication, and, therefore, it may represent an attractive therapeutic target.

Following work from Di Micco et al. (Di Micco et al., Front. Chem. 2021, 8:628609, has demonstrated that the N3 and 13b molecules present structural motifs similar to larazotide acetate, also called AT1001. AT1001 was hence investigated as a potential new inhibitor of M^{pro} enzyme. The authors have performed molecular modeling studies including docking and predictions using MM-GBSA predictions showing that AT1001 acetate docks extremely well in the catalytic domain of the M^{pro} enzyme without showing unfavorable steric interactions.

However, within this context, there remains a strong need to develop effective and safe therapeutic options for the prevention and/or treatment of viral infections, particularly infections of the respiratory tract such as SARS-COV-2 infections. Indeed, due to the high diffusion rate of SARS-COV-2 infection and the high frequency of complications due to ALI or ARDS, there is an incumbent risk of a high number of patients requiring hospitalization and subsequent assisted ventilation that may exceed the capacity of public health care systems to provide adequate assistance.

To date, there are still no proven therapies available to effectively treat COVID-19 infection. The discovery of a cure is urgently warranted but a potential breakthrough is limited by time-consuming processes traditionally needed to develop new drugs or vaccines.

### SUMMARY OF THE INVENTION

The technical problem posed and solved by the authors of the present invention is to provide novel compounds exhibiting a significant antiviral activity. The solution to this problem is represented by a peptide aldehyde of formula (1′), or a derivative, salt or stereoisomer thereof, according to claim 1.

By means of computer-aided design and in vitro experiments, the authors of the present invention have surprisingly identified a library of peptide derivatives of the drug larazotide, namely peptide aldehydes of formula (1):

(X)ₙ—R₂—R₁—H (formula 1)
or a derivative, salt or stereoisomer thereof wherein:
H is hydrogen;
n is an integer value ranging from 0 to 6;
R₁ is a single amino acid residue of valine, histidine, substituted alanine selected from 3-anthraniloyl-alanine or 2-naphtylalanine, substituted phenylalanine selected from para-benzoylphenylalanine (Bpa), para-biphenylalanine or p-chlorophenylalanine, tyrosine, tryptophan, serine, threonine, substituted glycine selected from allylglycine or α-Phenylglycine, or any other natural or non-natural amino acid or peptidomimetic scaffold;
R₂ is a single amino acid residue of valine, histidine, substituted alanine selected from 3-anthraniloyl-alanine or 2-naphtylalanine, phenylalanine or substituted phenylalanine selected from para-benzoylphenylalanine (Bpa), para-biphenylalanine or p-chlorophenylalanine, tyrosine, tryptophan, substituted glycine selected from allylglycine or α-Phenylglycine, or any other natural or non-natural amino acid or peptidomimetic scaffold;
X represents one or more amino acid residues each independently selected from glycine, proline or substituted proline selected from thioproline or trans-4-tert-Butoxy-L-proline, (α-methyl)alanine, or any other natural or non-natural amino acid or peptidomimetic scaffold; and/or from the group comprising N-(1,2,3,4-Tetrahydro-3-isoquinolinylcarbonyl) group, N-(2-Aminobenzoyl) group, or [(4-Fluorophenyl)sulfonyl] group;
wherein said one or more amino acid residues X optionally comprise an N-terminal protecting group;
and wherein said derivative of the peptide aldehyde of formula (1) is a cyclic derivative of a peptide of formula (1), or is a peptide of formula (1) in which the R₁—H group is replaced by a succinimide group, or is a peptide of formula (1) in which the aldehyde group is replaced by a reactive “warhead” group selected from: ketone; Michael acceptor, α-ketoamide; nitrile; bisulfite; electrophilic heterocycles; boronic acids; cyanamide; isothiocyanate; S-electrophile; electron-deficient (hetero)arene; vinyl sulfone and vinyl sulphonamide; acrylamide; propiolamide; N-methyl isoxazolium; fumaric acid ester; allenamide; propionitrile; alkenyl-/alkynyl-substituted heteroarene; haloalkane; α-halomethyl amide/ester/ketone; epoxide; aziridine; nitroalkane; α-cyanoacrylamide; α-cyanoenone; electron-deficient (hetero)arene with leaving group; acyloxymethylketone; hydroxymethyl and alkoxymethylketone; nonactivated terminal alkynes.

One particular aspect of the invention relates to a peptide aldehyde of formula (1′),

R₃—R₂—R₁—H
or a derivative, salt or stereoisomer thereof wherein:
H is hydrogen;
R₁ is a single amino acid residue of valine, histidine, substituted alanine selected from 3-anthraniloyl-alanine or 2-naphtylalanine, substituted phenylalanine selected from para-benzoylphenylalanine (Bpa), para-biphenylalanine or p-chlorophenylalanine, tyrosine, tryptophan, serine, threonine, or substituted glycine selected from allylglycine or α-Phenylglycine;
R₂ is a single amino acid residue of valine, histidine, substituted alanine selected from 3-anthraniloyl-alanine or 2-naphtylalanine, phenylalanine or substituted phenylalanine selected from para-benzoylphenylalanine (Bpa), para-biphenylalanine or p-chlorophenylalanine, tyrosine, tryptophan, or substituted glycine selected from allylglycine or α-Phenylglycine;

R₃ is a single amino acid residue of glycine, proline or substituted proline selected from thioproline or trans-4-tert-Butoxy-L-proline, (α-methyl)alanine, or is a group selected from N-(1,2,3,4-Tetrahydro-3-isoquinolinylcarbonyl) group, N-(2-Aminobenzoyl) group, or [(4-Fluorophenyl)sulfonyl] group;
wherein said R₃ single amino acid residue optionally comprises an N-terminal protecting group;
   and wherein said derivative of the peptide aldehyde of formula (1′) is a peptide of formula (1′) in which the R₁—H group is replaced by a succinimide group, or is a peptide of formula (1′) in which the aldehyde group is replaced by a reactive “warhead” group selected from: ketone; Michael acceptor, α-ketoamide; nitrile; bisulfite; electrophilic heterocycles; boronic acids; cyanamide; isothiocyanate; S-electrophile; electron-deficient (hetero)arene; vinyl sulfone and vinyl sulphonamide; acrylamide; propiolamide; N-methyl isoxazolium; fumaric acid ester; allenamide; propionitrile; alkenyl-/alkynyl-substituted heteroarene; haloalkane; α-halomethyl amide/ester/ketone; epoxide; aziridine; nitroalkane; α-cyanoacrylamide; α-cyanoenone; electron-deficient (hetero)arene with leaving group; acyloxymethylketone; hydroxymethyl and alkoxymethylketone; nonactivated terminal alkynes.

The compounds of the invention surprisingly exhibit a significant antiviral activity despite the shorter length and varied features of the new inserted residues in terms of size, polarity, and donor/acceptor H-bond groups with respect to larazotide. Notably, the authors of the invention have found that the aldehydic “warhead” of the compounds of formula (I) is able to establish a reversible covalent bond with Cys145 of the main protease enzyme of SARS-COV-2, thereby increasing the binding affinity vs. the 30 macromolecule.

The identified compounds possess several advantages over the molecules already known from the prior art: particularly, because of the small size they can be more easily synthesized, obtained in highly pure form, and can be readily delivered in a wide variety of modalities, particularly by way of aerosol, nebulization or spray-based intra-nasal administration. The latter mucosal administration route has the advantage to be local and non-systemic, improving the safety profile of the antiviral drug despite the common therapeutics against infections. The latter mucosal administration increments the patient compliance and allows outpatient administering with no need for hospitalization. Moreover, the smaller size of the molecules should ameliorate the membrane permeability than the parent octapeptide larazotide. These peptides should be effective against the current developing variants of SARS-COV-2, as they target unmuted enzyme fundamental for viral life cycle. Considering that high sequence and structural similarity of M^{pro} of difference SARS viruses, the proposed compounds could be applied for treatment of COVID-19 similar viruses.

Therefore, object of the invention are compounds of formula (1′), pharmaceutical compositions comprising them, as well as uses thereof in the prevention and/or treatment of viral infections.

Preferred features of the present invention are the object of the dependent claims. Additional features and advantages of the various aspects of the present invention will become apparent from the following detailed description of its preferred embodiments, together with the accompanying figures. Preferred embodiments are intended to explain and exemplify certain aspects of the present of invention but should not be construed as limiting its scope of protection.

### BRIEF DESCRIPTION OF THE DRAWINGS

The present invention and the following detailed description of preferred embodiments thereof may be better understood with reference to the following figures:

FIG. 1: Three-dimensional model of the AT1001-M^{pro} interactions.

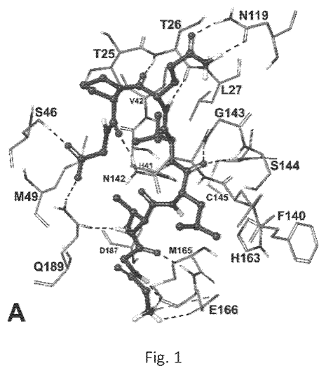

### GLOSSARY

Unless defined otherwise, all technical and scientific terms used herein have the same meaning as commonly understood by those of ordinary skill in the art to which the invention belongs.

The expression “amino acid residue” as used herein, indicates a group with a structure like —NH—CHR—CO— written with the N-terminal at the left and the C-terminal at the right. An amino acid residue can be abbreviated using standard one-letter or three-letter codes such as the following: alanine (“A” or “Ala”), Arginine (“R” or “Arg”), Asparagine (“N” or “Asn”), Aspartic acid (“D” or “Asp”), Cysteine (“C” or “Cys”), Glutamine (“Q” or Gln”), Glutamic acid (“E” or “Glu), Glycine (“G” or “Gly”), Histidine (“H” or “His”), Isoleucine (“I” or “He”), Leucine (“L” or “Leu”), Lysine (“K” or “Lys”), Methionine (“M” or “Met”), Phenylalanine (“F” or “Phe”), Proline (“P” or “Pro”), Serine (“S” or “Ser”), Threonine (“T” or “Thr”), Tryptophan (“W” or “Trp”), Tyrosine (“Y” or “Tyr”) and Valine (“V” or “Val”).

Structures depicted herein are also meant to include any non-naturally occurring amino acids. The expression “non-naturally occurring amino acids” is intended to comprise D- and L-forms of amino acids other than those commonly found in nature as well as modified naturally occurring amino acids. Non limiting examples of useful non-naturally occurring amino acids are: D/L-Kynurenine, D/L-Hyp(tBu)—OH, D/L-Bpa-OH, D/L-Bip-OH, D/L-2Nal-OH, D/L-t-butylglycine, D/L-Abu-OH, D/L-Aib-OH, D/L-Phe(4-NH₂)—OH, D/L-Phe(4-NO₂)—OH, D/L-Phe(4-Cl)—OH, D/L-6-Ahx-OH, D/L-Orn-OH, D/L-Hse-OH, D/L-Cha-OH, D/L-Chg-OH, D/L-Cit-OH, D/L-Nva-OH, D/L-Phg-OH, D/L-Tic-OH, D/L-Nle-OH, D/L-Sar-OH.

The abbreviation “—H” used in the formulas disclosed in the present specification denotes an aldehyde derivative of an amino acid residue, meaning that the C-terminal end of the amino acid residue is converted from a carboxylic group to an aldehyde group. All amino acids in the peptide aldehydes of the invention can also be in both D- or L-form.

Unless otherwise stated, structures depicted herein are also meant to include all isomeric (e.g. enantiomeric, diastereomeric, and geometric, or conformational) forms of the structure; for example the R and S configurations for each asymmetric centre, (Z) and (E) double bond isomers as well as enantiomeric, diastereomeric, and geometric (or conformational) mixtures of the present compounds are within the scope of the invention. Unless otherwise stated, all tautomeric forms of the compounds of the invention are within the scope of the invention.

The term “stereoisomer”, as used herein, is a general term for all isomers of individual molecules that differ only in the orientation of their atoms in space. It includes mirror image isomers (enantiomers), geometric (cis/trans) isomers, and isomers of compounds with more than one chiral center that are not mirror images of one another (diastereoisomers).

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of the invention. Such compounds are useful, for example, as analytical tools or probes in biological assays, or as therapeutic agents.

The expression “peptidomimetic scaffold” as used herein is referred to chemical structures endowed with a pharmacophore mimicking a natural amino acid, peptide or protein in the three-dimensional conformational space and preserving the interaction with the biological target and giving rise to the same biological effect.

The expression “optionally substituted” can be used interchangeably with the phrase “substituted or unsubstituted”. In general, the term “substituted”, whether preceded by the term “optionally” or not, refers to the replacement of hydrogen atoms in a given structure with the specific substituent. Specific substituted compounds are disclosed in the description of the invention and examples thereof.

As used herein, an “effective amount” is defined as the amount required to confer a therapeutic, protective or preventive effect on the treated subject, and is typically determined based on age, surface area, weight, and condition of the subject.

As used herein, “subject” refers to a mammal, preferably a human. In some embodiments, said “subject” is preferably an individual suffering from or at risk of developing a viral infection or a disease caused by or associated to a viral infection, and particularly a SARS-COV-2 infection. Hence, in some embodiments, a subject can be an individual that has been diagnosed as being affected by or as being at risk of suffering from a viral infection and/or a disease caused by or associated to a viral infection.

As used herein, the term “SARS-COV-2” refers to “severe acute respiratory syndrome coronavirus 2”, a viral strain of the SARS-related Coronavirus species, belonging to the family of Coronaviridae and responsible for Coronavirus disease 2019 (COVID-19).

The outermost layer of the SARS-COV-2 virus is characterized by a coating of S glycoproteins assembled to form trimeric structures that constitute the characteristic “crown” that surrounds the virion.

As used herein, the term “main protease” or M^{pro}, also termed 3CL protease, refers to a 33.8-kDa cysteine protease which mediates the maturation of functional polypeptides involved in the assembly of replication-transcription machinery in Coronavirus. M^{pro} has no human homolog and is highly conserved among all Coronaviruses. Structure of M^{pro} from SARS-COV-2 revealed a three-domain (domains I to III) architecture which is conserved among Coronaviruses. In the cleft between domains I and II, it features a non-canonical Cys-His dyad as the catalytic site. The cysteine residue of the Cys-His dyad undergoes nucleophilic attack on the reactive atom of the substrate, while the histidine residue helps to stabilize the intermediate state. Around this dyad, M^{pro} forms a conserved binding pocket which is composed of four subsites (S₁′, S₁, S₂, and S₄) well accommodating the substrate.

In any point of the present specification, the terms “comprising” or “comprises” may be replaced by the terms “consisting of” or “consists of”.

### DETAILED DESCRIPTION OF PREFERRED EMBODIMENTS

In the following, several embodiments of the invention will be described. It is intended that the features of the various embodiments can be combined, where compatible. In general, subsequent embodiments will be disclosed only with respect to the differences with the previously described ones.

As previously mentioned, a first object of the present invention is represented by a peptide aldehyde of the formula (1):

(X)ₙ—R₂—R₁—H (formula 1)
or a derivative, salt or stereoisomer thereof wherein:
H is hydrogen;
n is an integer value ranging from 0 to 6;
R₁ is a single amino acid residue of valine, histidine, substituted alanine selected from 3-anthraniloyl-alanine or 2-naphtylalanine, phenylalanine, substituted phenylalanine selected from meta-hydroxyphenylalanine, p-bromophenylalanine, para-benzoylphenylalanine (Bpa), para-biphenylalanine or p-chlorophenylalanine, tyrosine, tryptophan, serine, threonine, asparagine, glutamine, substituted glycine selected from cyclohexylglycine, allylglycine or α-Phenylglycine, or any other natural or non-natural amino acid or peptidomimetic scaffold;
R₂ is a single amino acid residue of valine, serine, histidine, substituted alanine selected from 3-anthraniloyl-alanine or 2-naphtylalanine, phenylalanine or substituted phenylalanine selected from para-benzoylphenylalanine (Bpa), para-biphenylalanine or p-chlorophenylalanine, tyrosine, threonine, tryptophan, substituted glycine selected from cyclohexyl glycine, allylglycine or α-Phenylglycine, or any other natural or non-natural amino acid or peptidomimetic scaffold;
X represents one or more amino acid residues each independently selected from glycine, proline or substituted proline selected from thioproline or trans-4-tert-Butoxy-L-proline, (α-methyl)alanine, or any other natural or non-natural amino acid or peptidomimetic scaffold; and/or from the group comprising N-(1,2,3,4-Tetrahydro-3-isoquinolinylcarbonyl) group, N-(2-Aminobenzoyl) group, or [(4-Fluorophenyl)sulfonyl] group;
wherein said one or more amino acid residues X optionally comprise an N-terminal protecting group;
and wherein said derivative of the peptide aldehyde of formula (1) is a cyclic derivative of a peptide of formula (1), or is a peptide of formula (1) in which the R₁—H group is replaced by a succinimide group, or is a peptide of formula (1) in which the aldehyde group is replaced by a reactive “warhead” group selected from: ketone; Michael acceptor, α-ketoamide; nitrile; bisulfite; electrophilic heterocycles; boronic acids; cyanamide; isothiocyanate; S-electrophile; electron-deficient (hetero)arene; vinyl sulfone and vinyl sulphonamide; acrylamide; propiolamide; N-methyl isoxazolium; fumaric acid ester; allenamide; propionitrile; alkenyl-/alkynyl-substituted heteroarene; haloalkane; α-halomethyl amide/ester/ketone; epoxide; aziridine; nitroalkane; α-cyanoacrylamide; α-cyanoenone; electron-deficient (hetero)arene with leaving group; acyloxymethylketone; hydroxymethyl and alkoxymethylketone; nonactivated terminal alkynes.

According to a preferred aspect of the invention, the peptide aldehyde of formula (1) hence comprises a minimum of two amino acid residues up to a maximum of eight amino acid residues, preferably it comprises three amino acid residues.

In one embodiment of the invention, the peptide aldehyde of formula (1) is an octapeptide aldehyde, preferably is the aldehyde of the octapeptide larazotide, which larazotide is also known as AT1001 (PubChem CID: 9810532; CAS Number: 258818-34-7), namely is the octapeptide larazotide wherein the C-terminal glycine residue is modified to bear an aldehyde group “—H” or in which said aldehyde group “—H” is replaced by a reactive “warhead” group selected from: ketone; Michael acceptor, α-ketoamide; nitrile; bisulfite; electrophilic heterocycles; boronic acids; cyanamide; isothiocyanate; S-electrophile; electron-deficient (hetero)arene; vinyl sulfone and vinyl sulphonamide; acrylamide; propiolamide; N-methyl isoxazolium; fumaric acid ester; allenamide; propionitrile; alkenyl-/alkynyl-substituted heteroarene; haloalkane; α-halomethyl amide/ester/ketone; epoxide; aziridine; nitroalkane; α-cyanoacrylamide; α-cyanoenone; electron-deficient (hetero)arene with leaving group; acyloxymethylketone; hydroxymethyl and alkoxymethylketone; nonactivated terminal alkynes.

Another aspect of the invention is related to a peptide aldehyde of formula (1′),

R₃—R₂—R₁—H
or a derivative, salt or stereoisomer thereof wherein:
H is hydrogen;

R₁ is a single amino acid residue of valine, histidine, substituted alanine selected from 3-anthraniloyl-alanine or 2-naphtylalanine, phenylalanine, substituted phenylalanine selected from meta-hydroxyphenylalanine, p-bromophenylalanine, para-benzoylphenylalanine (Bpa), para-biphenylalanine or p-chlorophenylalanine, tyrosine, tryptophan, serine, threonine, asparagine, glutamine, or substituted glycine selected from cyclohexylglycine, allylglycine or α-Phenylglycine;

R₂ is a single amino acid residue of valine, serine, histidine, substituted alanine selected from 3-anthraniloyl-alanine or 2-naphtylalanine, phenylalanine or substituted phenylalanine selected from para-benzoylphenylalanine (Bpa), para-biphenylalanine or p-chlorophenylalanine, tyrosine, threonine, tryptophan, or substituted glycine selected from cyclohexyl glycine, allylglycine or α-Phenylglycine;

R₃ is a single amino acid residue of glycine, substituted glycine selected from cyclohexyl glycine and phenylglycine, valine, 3-methylvaline, proline or substituted proline selected from thioproline or trans-4-tert-Butoxy-L-proline, leucine, isoleucine, alanine, (α-methyl)alanine, or is a group selected from N-(1,2,3,4-Tetrahydro-3-isoquinolinylcarbonyl) group, N-(2-Aminobenzoyl) group, or [(4-Fluorophenyl)sulfonyl] group;
wherein said R₃ single amino acid residue optionally comprises an N-terminal protecting group;
and wherein said derivative of the peptide aldehyde of formula (1′) is a peptide of formula (1′) in which the R₁—H group is replaced by a succinimide group, or is a peptide of formula (1′) in which the aldehyde group is replaced by a reactive “warhead” group selected from: ketone; Michael acceptor, α-ketoamide; nitrile; bisulfite; electrophilic heterocycles; boronic acids; cyanamide; isothiocyanate; S-electrophile; electron-deficient (hetero)arene; vinyl sulfone and vinyl sulphonamide; acrylamide; propiolamide; N-methyl isoxazolium; fumaric acid ester; allenamide; propionitrile; alkenyl-/alkynyl-substituted heteroarene; haloalkane; α-halomethyl amide/ester/ketone; epoxide; aziridine; nitroalkane; α-cyanoacrylamide; α-cyanoenone; electron-deficient (hetero)arene with leaving group; acyloxymethylketone; hydroxymethyl and alkoxymethylketone; nonactivated terminal alkynes; or is a peptide of formula R₂—R₁—H (1″) wherein R₁ is histidine and R₂ is phenylalanine comprising 1H-Pyrrol-2-carbonyl as N-terminal protecting group.

One preferred embodiment of the invention is referred to a peptide aldehyde of the formula (1′):

(X)ₙ—R₂—R₁—H (formula 1)
or a derivative, salt or stereoisomer thereof wherein:
H is hydrogen;
R₁ is a single amino acid residue of valine, histidine, substituted alanine selected from 3-anthraniloyl-alanine or 2-naphtylalanine, substituted phenylalanine selected from para-benzoylphenylalanine (Bpa), para-biphenylalanine or p-chlorophenylalanine, tyrosine, tryptophan, serine, threonine, or substituted glycine selected from allylglycine or α-Phenylglycine;
R₂ is a single amino acid residue of valine, histidine, substituted alanine selected from 3-anthraniloyl-alanine or 2-naphtylalanine, phenylalanine or substituted phenylalanine selected from para-benzoylphenylalanine, para-biphenylalanine or p-chlorophenylalanine, tyrosine, tryptophan, or substituted glycine selected from allylglycine or α-Phenylglycine;

R₃ is a single amino acid residue of glycine, proline or substituted proline selected from thioproline or trans-4-tert-Butoxy-proline, (α-methyl)alanine, or is a group selected from N-(1,2,3,4-Tetrahydro-3-isoquinolinylcarbonyl) group, N-(2-Aminobenzoyl) group, or [(4-Fluorophenyl) sulfonyl] group;
wherein said R₃ single amino acid residue optionally comprises an N-terminal protecting group; and wherein said derivative is a peptide of formula (1′) in which the R₁—H group is replaced by a succinimide group.

Each single amino acid residue in R₁, R₂ or R₃ may hence be a natural or non-naturally occurring alpha- or beta-amino acid selected among those listed above.

The N-terminal protecting group may be any amino-terminal protecting group which can be employed in peptide synthesis. Non limiting examples of suitable groups include formyl, acyl group (O(C═O)R), wherein R is a saturated C₁₋₁₆ alkyl chain, benzoyl, trifluoroacetyl, fluoro-methoxy carbonyl, methoxysuccinyl, aromatic urethane protecting groups, such as, benzyloxycarbonyl; t-butyloxycarbonyl (boc), adamantyloxycarbonyl, p-methoxybenzyl carbonyl (MOZ), benzyl (Bn), p-methoxybenzyl (PMB), p-methoxyphenyl (PMP), or fluorenylmethyloxycarbonyl.

Preferably, the N-terminal protecting group of a peptide of formula (1′), salt or stereoisomer thereof according to the present invention is selected from acyl group (O(C═O)R), wherein R is a saturated C₁₋₁₆ alkyl chain, 1-(1H-indol-2-yl)carbonyl, 1-(1H-indol-5-yl)carbonyl, 1H-pyrrol-3-carbonyl, 1H-pyrrol-2-carbonyl, 2-furancarbonyl, 2-(4-pyridinyl)-1,3-thiazole-4-carbonyl, cyclopent-3-ene-1-carbonyl, cyclohexanecarbonyl, cyclopentylacetyl, 1-(4-methylphenyl)cyclopropancarbonyl, cyclopropylcarbonyl, cycloesylacetyl, 1,3-thiazolidine-4-carbonyl, t-butyloxycarbonyl or fluorenylmethyloxycarbonyl, more preferably acetyl.

In one embodiment, the N-terminal protecting group of a peptide of formula (1′), salt or stereoisomer thereof according to the present invention is an acetyl group.

According to one embodiment, the peptide aldehyde of the present invention is a peptide aldehyde of formula (1′) wherein:
H is hydrogen;
R₁ is a single amino acid residue of valine, histidine, or tyrosine;
R₂ is a single amino acid residue of phenylalanine, histidine, or tyrosine;
R₃ is a single amino acid residue of glycine or trans-4-tert-Butoxy-L-proline, or is the N-(1,2,3,4-Tetrahydro-3-isoquinolinylcarbonyl) group, wherein said R₃ single amino acid residue optionally comprises an N-terminal protecting group selected from C₂₋₁₆ acyl, t-butyloxycarbonyl or fluorenylmethyloxycarbonyl.

According to another aspect of the invention, the peptide aldehyde is a peptide aldehyde of formula (1′) wherein:
H is hydrogen;
R₁ is a single amino acid residue of histidine;
R₂ is a single amino acid residue of phenylalanine, histidine, or tyrosine; and
R₃ is a single amino acid residue of glycine or is the N-(1,2,3,4-Tetrahydro-3-isoquinolinylcarbonyl) group, wherein said R₃ single amino acid residue optionally comprises an N-terminal protecting group selected from C₂₋₁₆ acyl, t-butyloxycarbonyl or fluorenylmethyloxycarbonyl.

In one preferred embodiment,
R₁ is a single amino acid residue of histidine;
R₂ is a single amino acid residue of phenylalanine; and
R₃ is a single amino acid residue of glycine; wherein said R₃ single residue optionally comprises an N-terminal protecting group selected from C₂₋₁₆ acyl, t-butyloxycarbonyl or fluorenylmethyloxycarbonyl.

Even more preferably,
R₁ is a single amino acid residue of histidine;
R₂ is a single amino acid residue of phenylalanine; and
R₃ is a single amino acid residue of glycine; wherein said R₃ single residue optionally comprises an acetylated N-terminal group.

In a preferred aspect of the invention the peptide aldehyde of the invention comprises three natural and/or non-naturally occurring amino acid residues.

According to one embodiment, the peptide aldehyde of formula (1) or (1′) of the invention is selected from the following compounds:

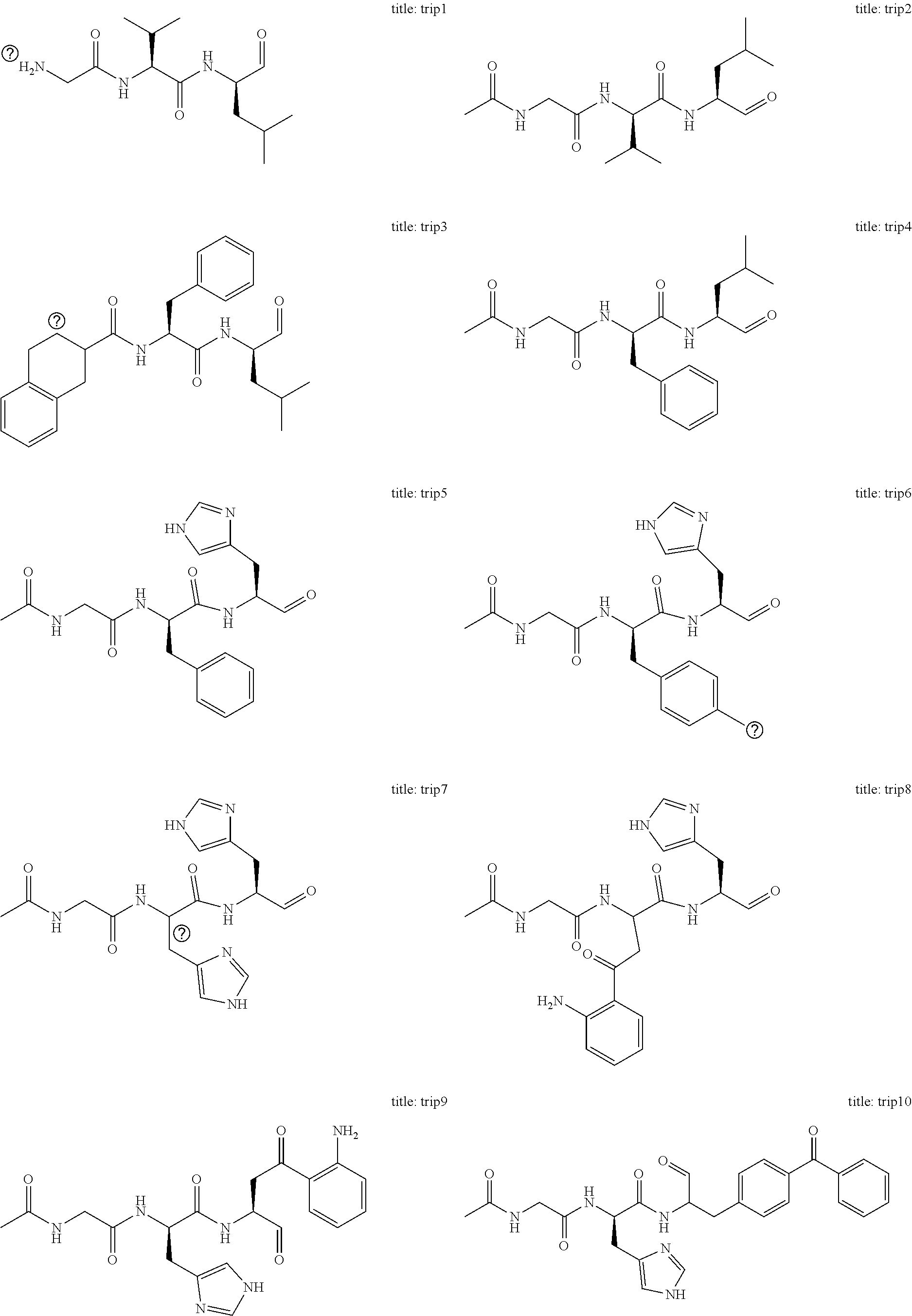

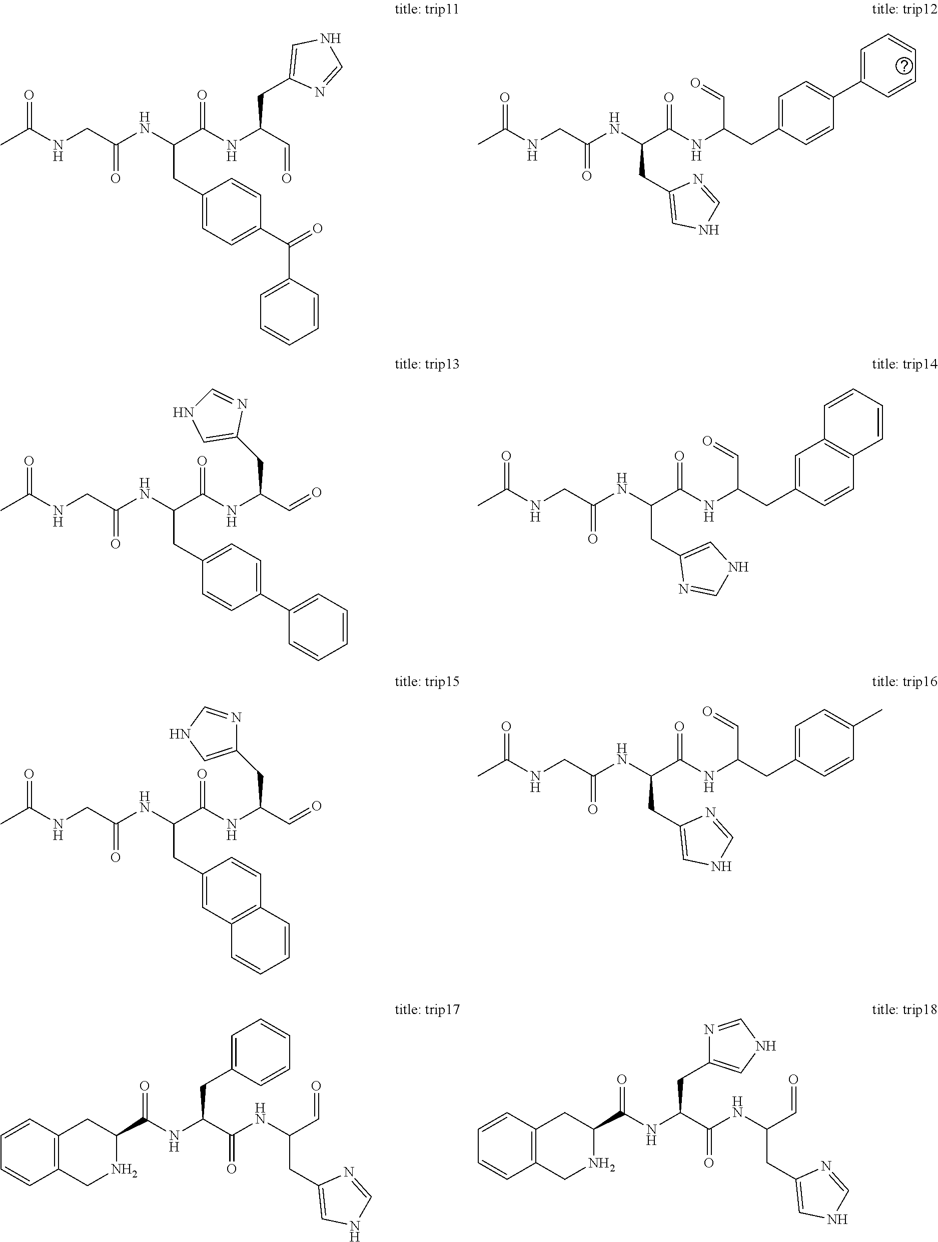

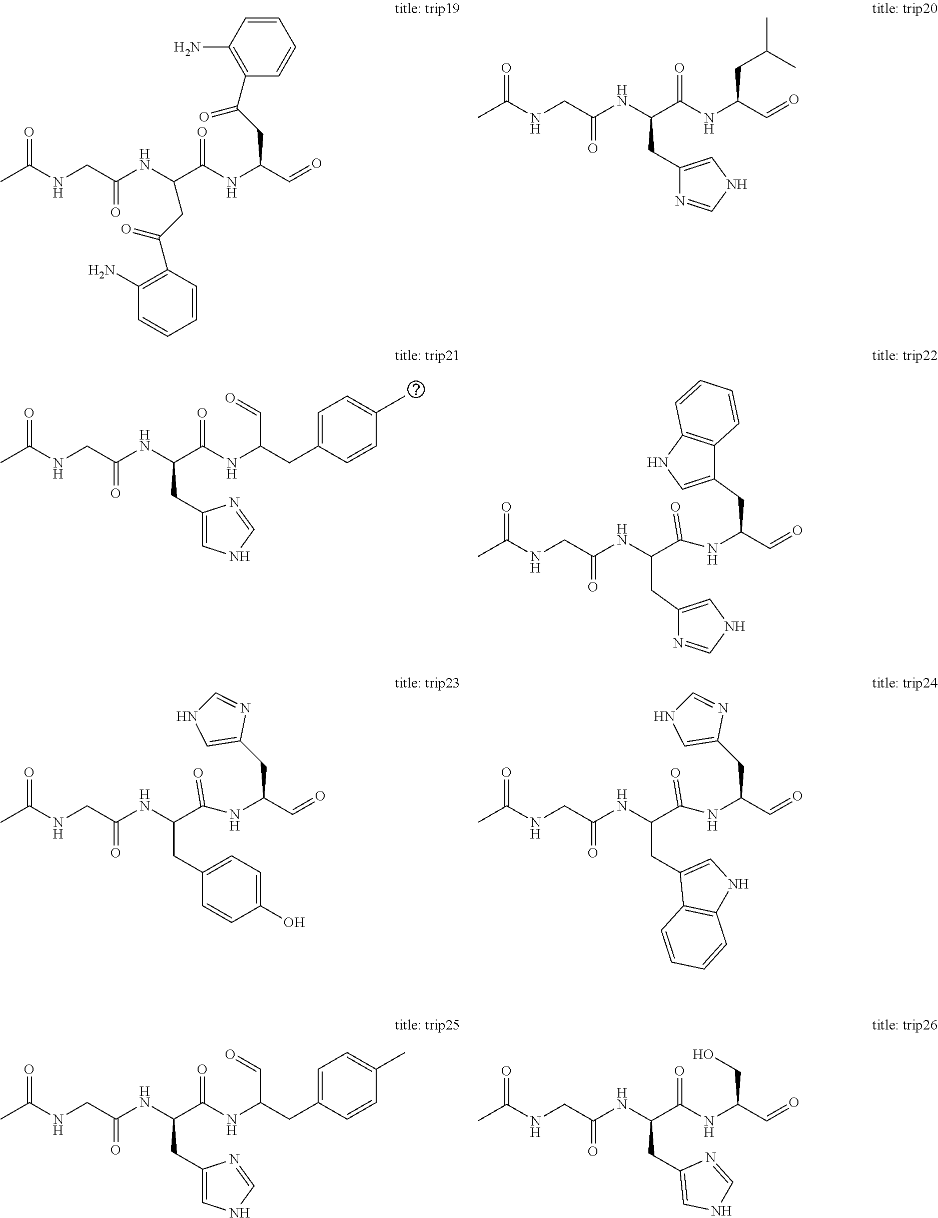

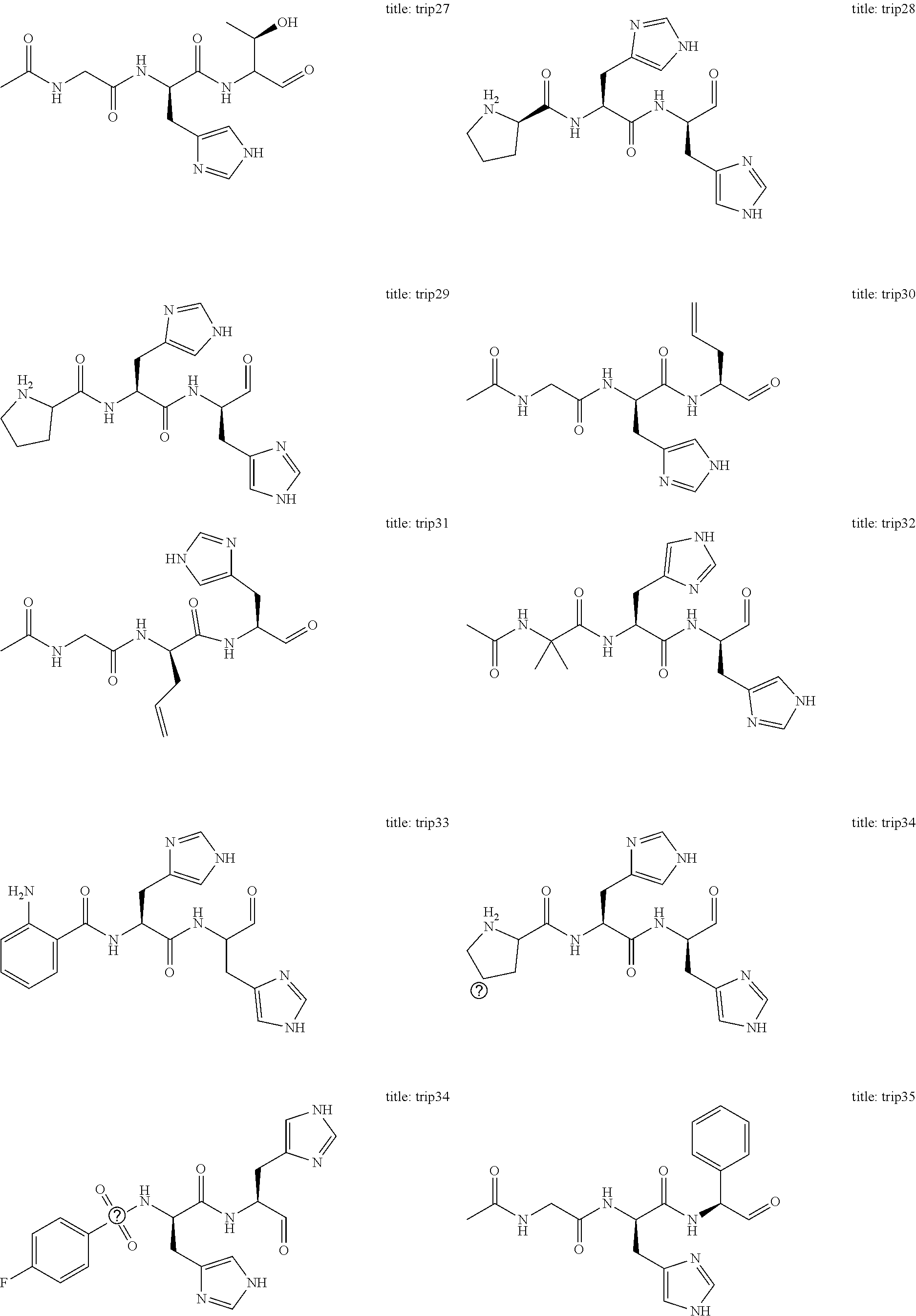

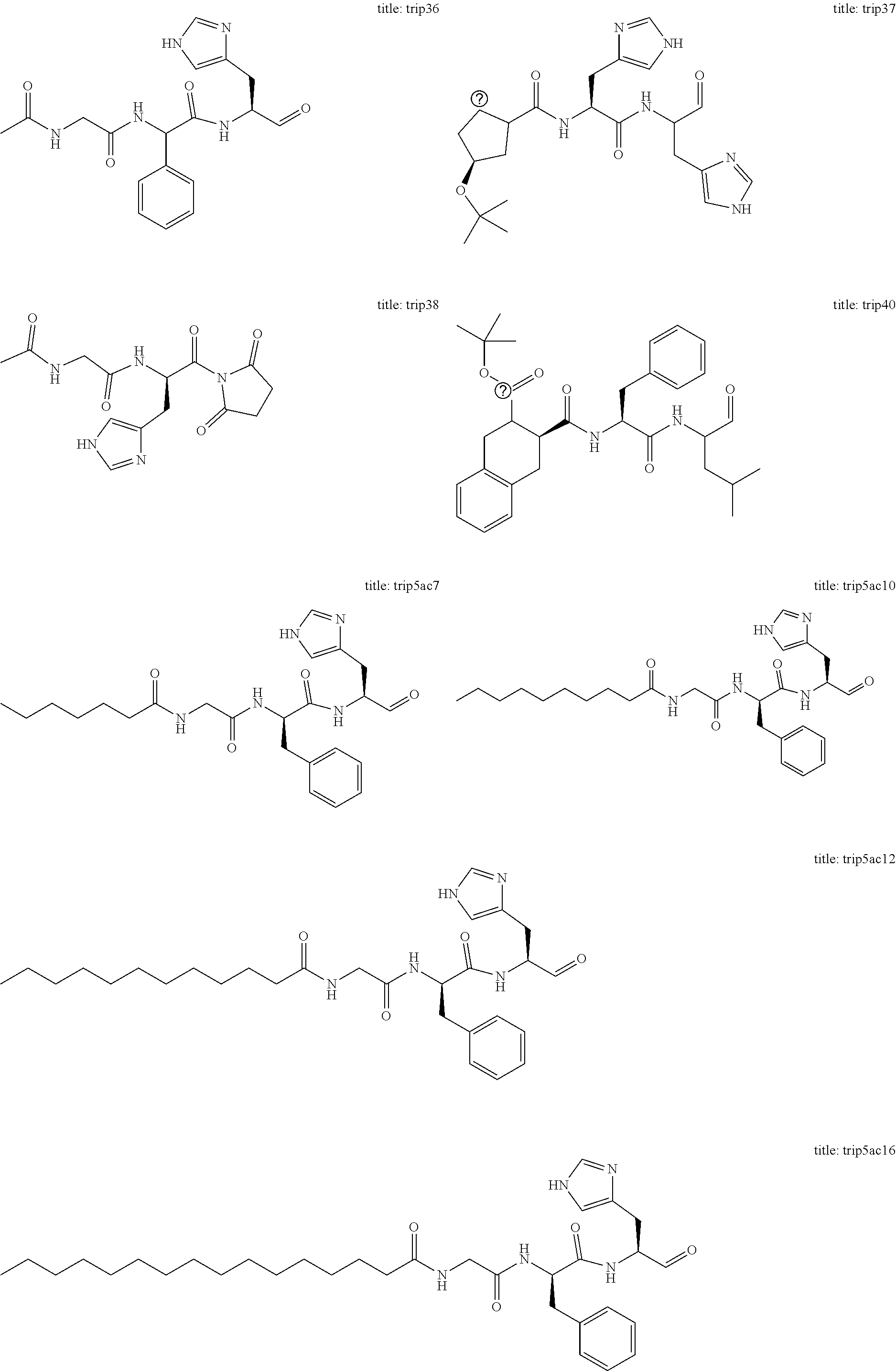

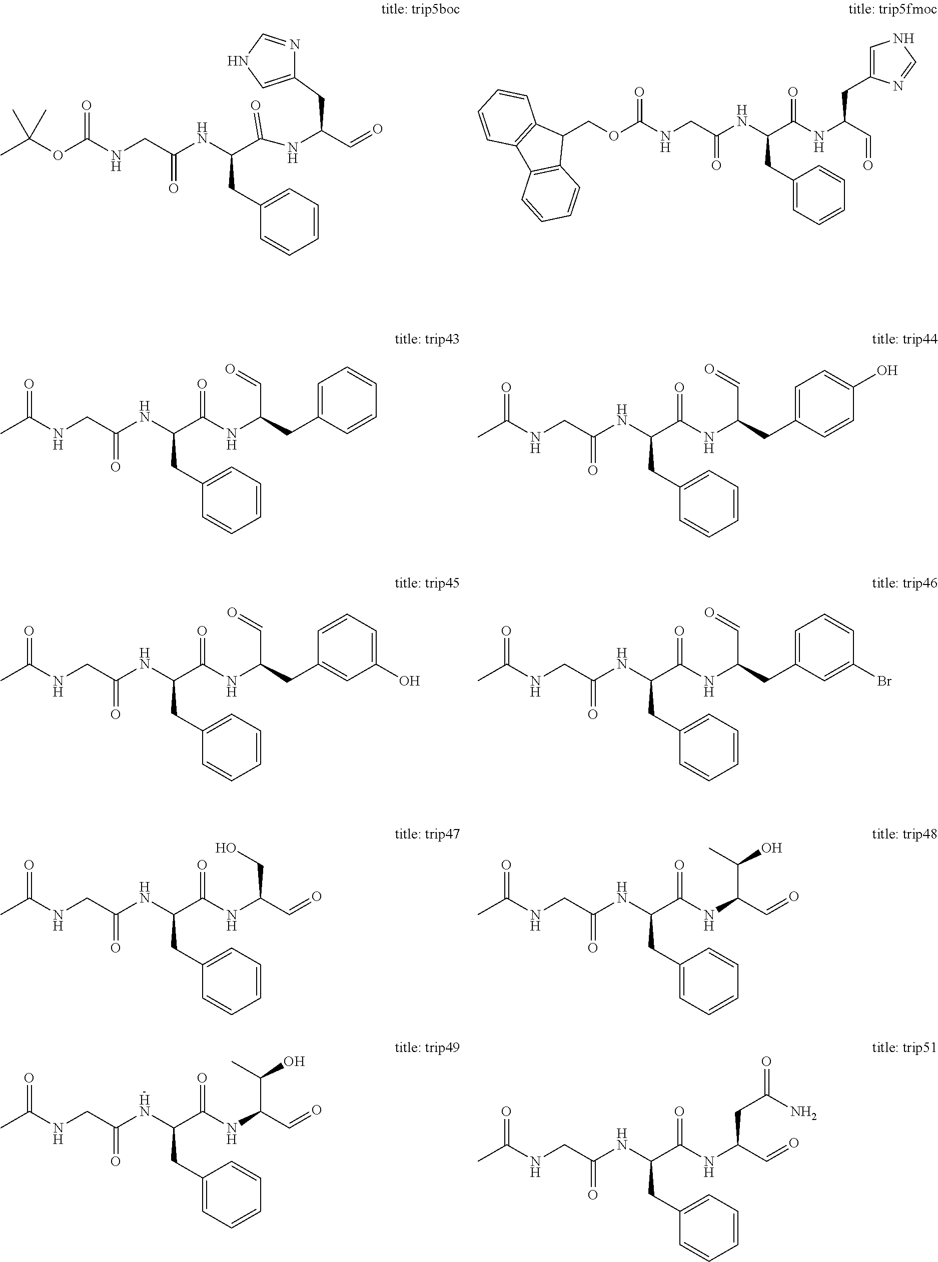

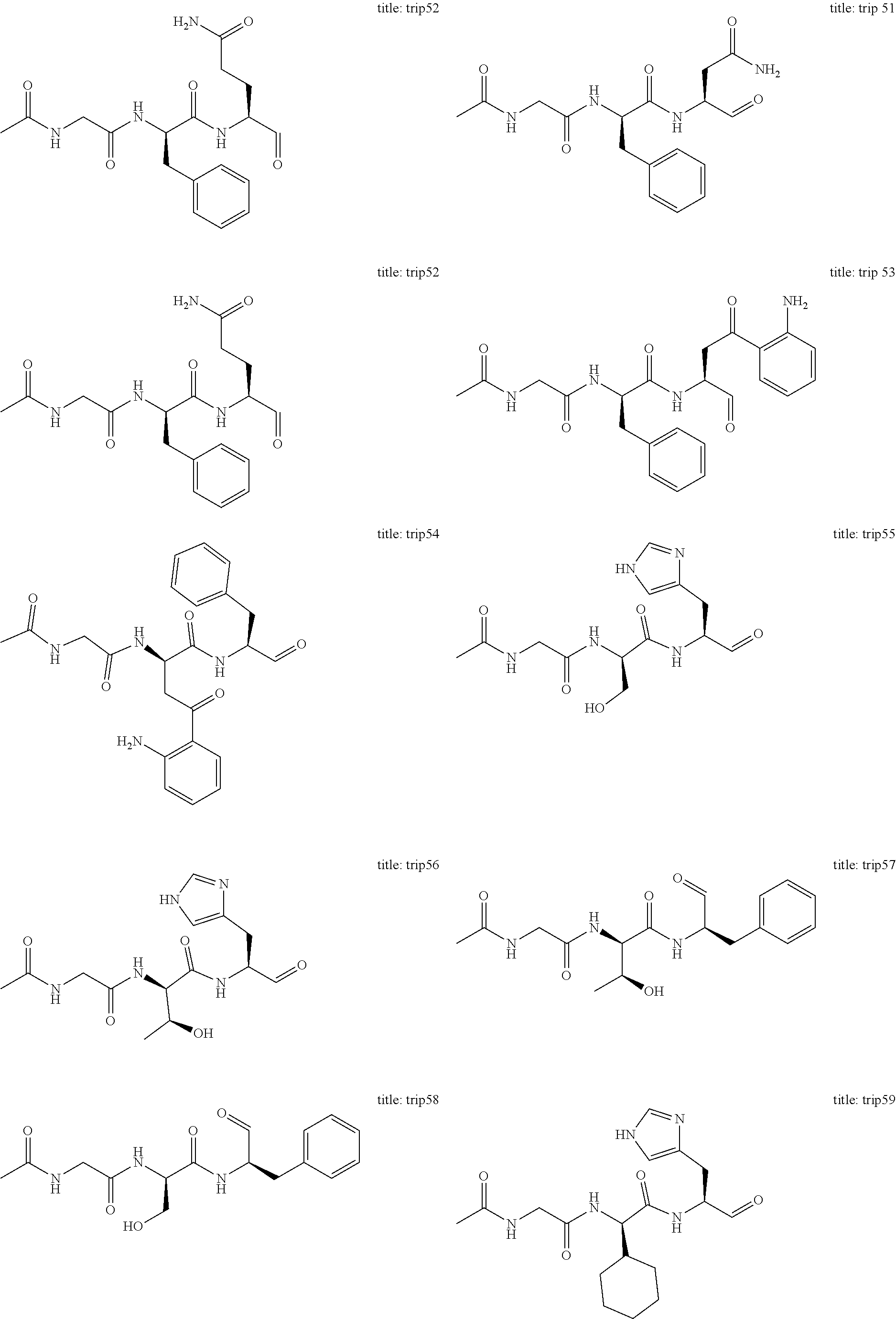

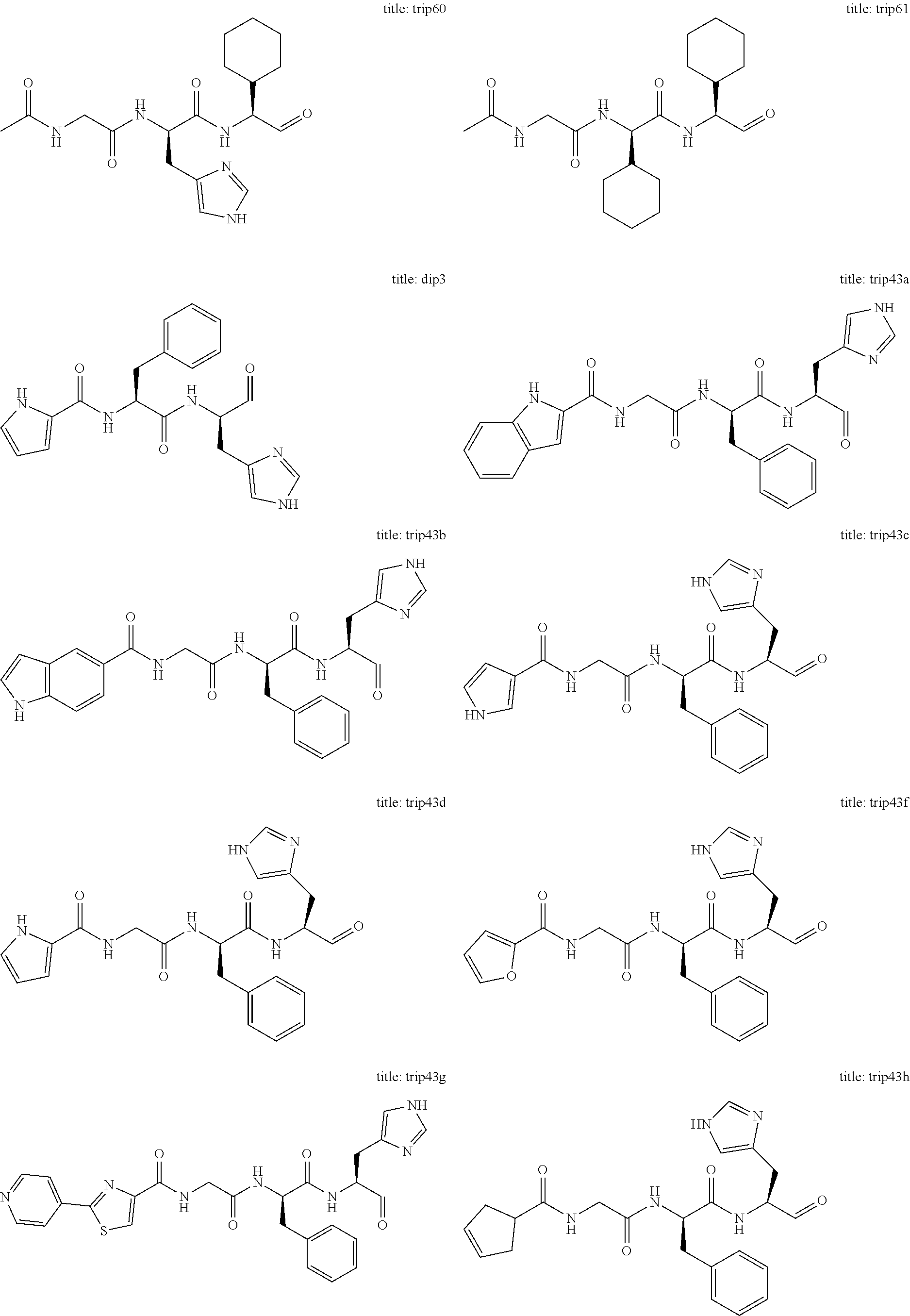

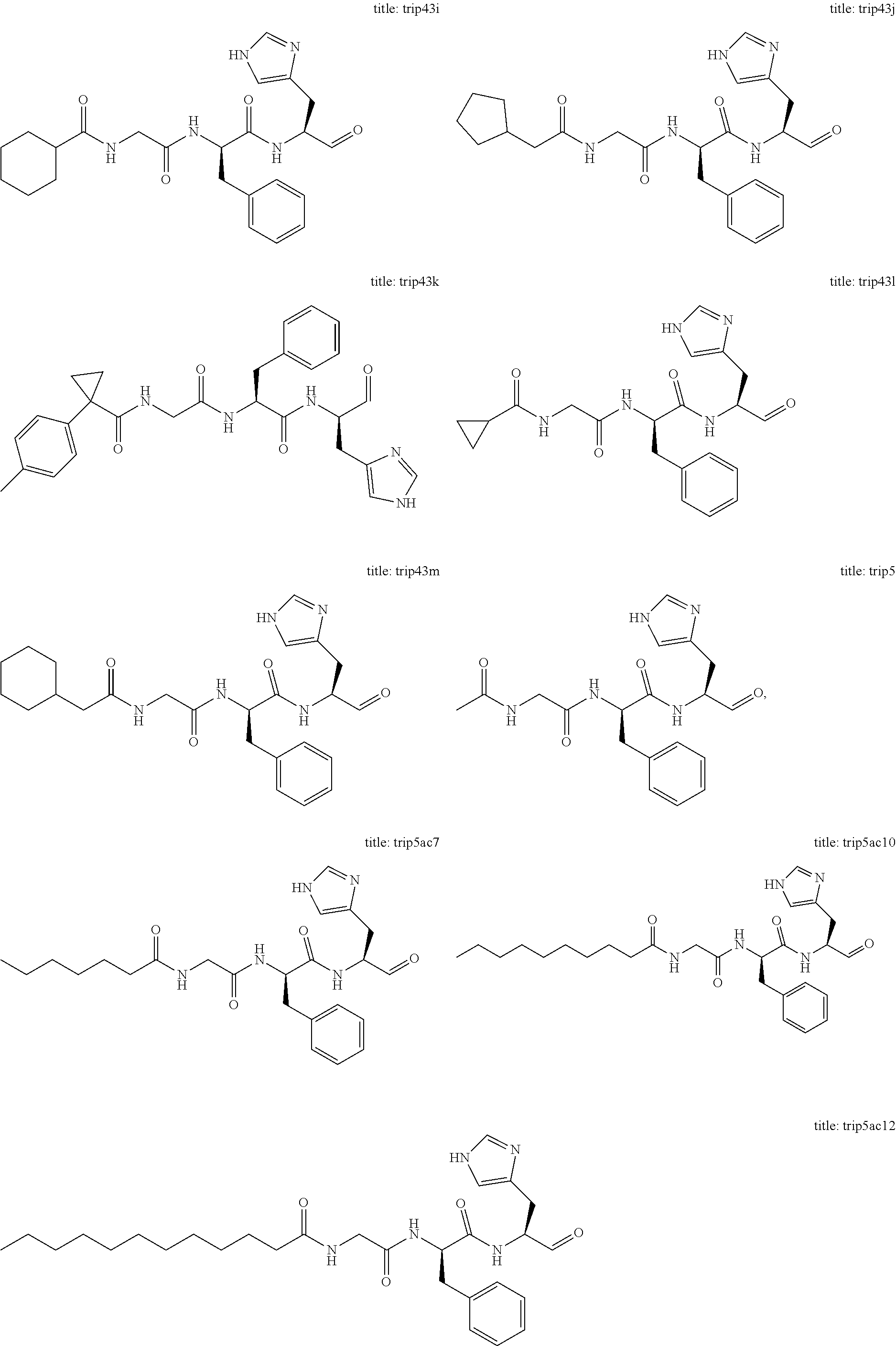

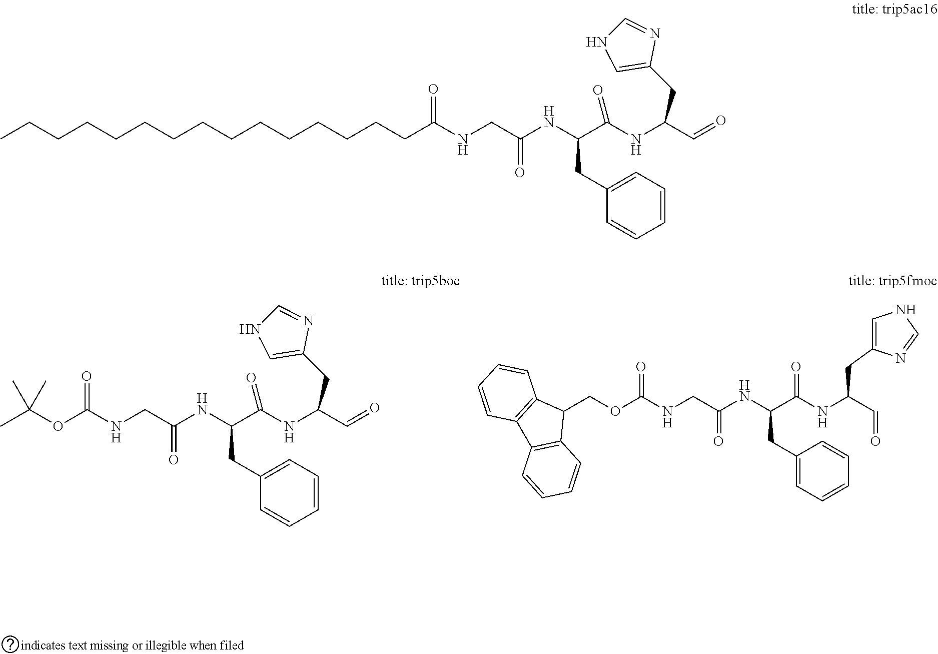 [SMILES: CCCCCCCCCCCCCCCC(=O)NCC(=O)N[C@H](Cc1ccccc1)C(=O)N[C@H](C=O)Cc1c[nH]cn1 ; CC(C)(C)OC(=O)NCC(=O)N[C@H](Cc1ccccc1)C(=O)N[C@H](C=O)Cc1c[nH]cn1 ; O=C[C@H](Cc1c[nH]cn1)NC(=O)[C@@H](Cc1ccccc1)NC(=O)CNC(=O)OCC1c2ccccc2-c2ccccc21]

Even more preferably, the peptide aldehyde of formula (1) or (1′) or a salt or stereoisomer thereof according to the present invention is a peptide aldehyde selected among the following structures:

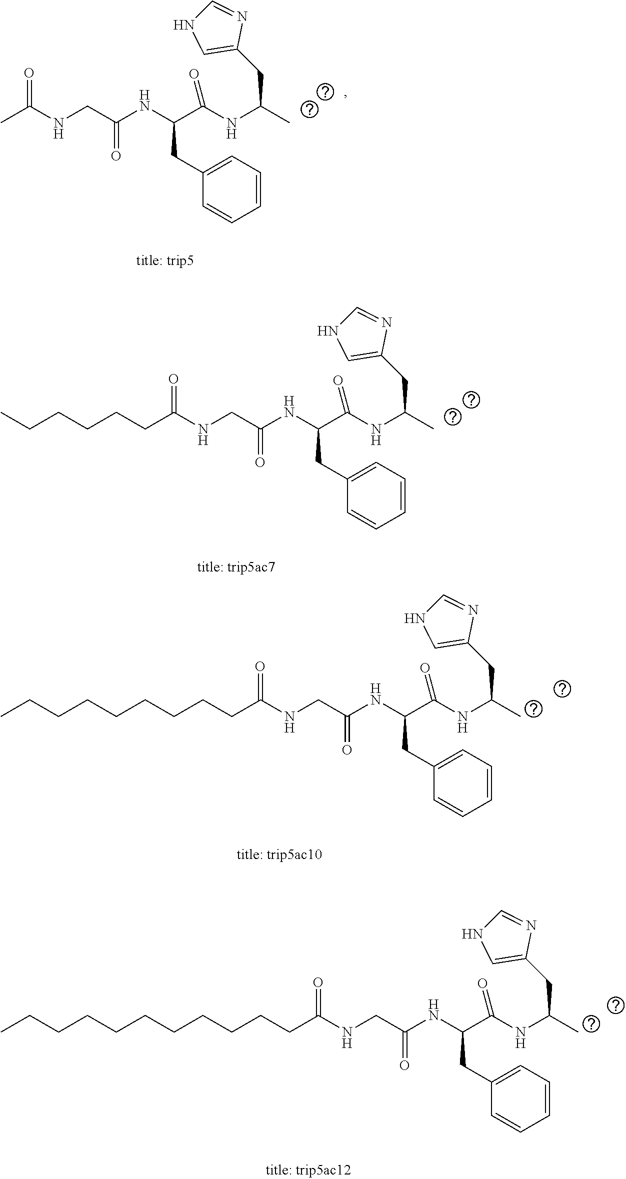

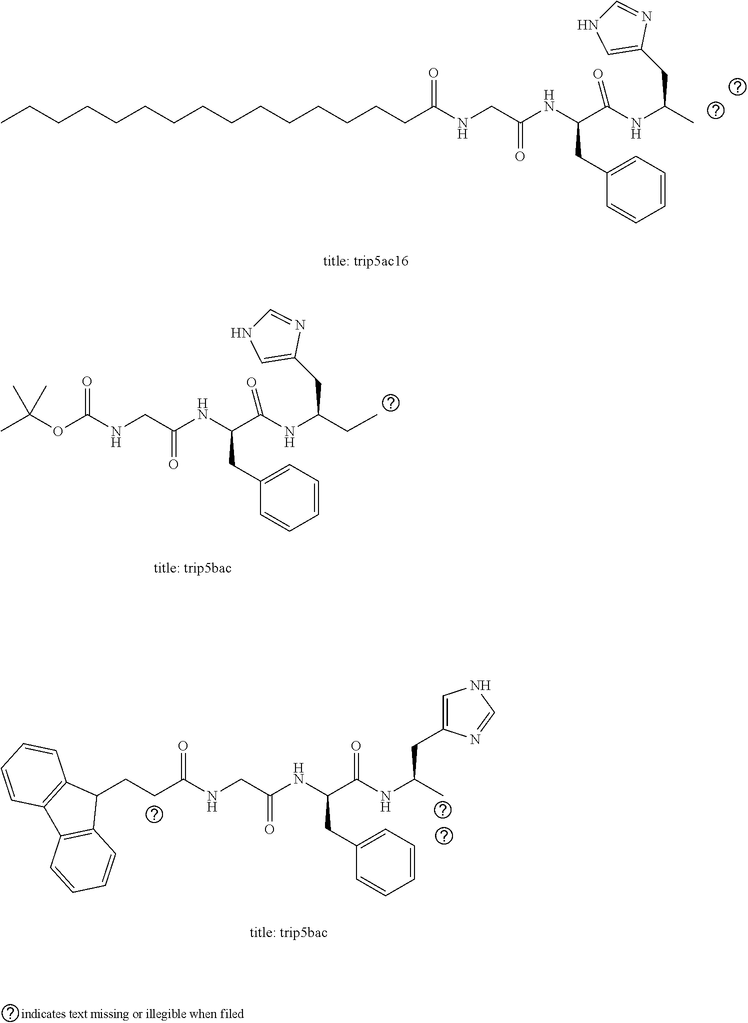

According to one aspect of the invention, the peptide aldehydes of formula (1) or (1′), or a derivative, salt or stereoisomer thereof according to any of the embodiments discloses in the present specification, exhibit antiviral activity.

The term “antiviral activity” as used herein is understood to refer to any effects useful as mechanisms for prevention or treatment of viral infections, such as an effect of suppressing viral replication, an effect of decreasing viral infections, and an effect of decreasing or eliminating infecting viruses. The skilled person is aware of means and methods to determine whether a peptide has an antiviral activity, including but not limited those methods shown in the Examples.

In one particular embodiment of the invention, the peptide aldehydes of formula (1) or (1′), or a derivative, salt or stereoisomer thereof according to any of the embodiments discloses in the present specification, are inhibitors of the main protease (M^{pro}) of Corovaviruses, preferably the M^{pro} of SARS-COV-2.

### Methods

The novel peptide aldehydes of formula (1) or (1′) according to any of the embodiments described in the present specification may be prepared in accordance with well-known procedures for preparing peptide aldehydes from their constituent residues. Although specific techniques for their preparation are described herein, it is to be understood that all appropriate techniques suitable for the production of the peptide aldehydes of formula (1) or (1′) are intended to be within the scope of this invention.

According to one embodiment, a peptide aldehyde of formula (1) or (1′) as described herein can be produced by chemical synthesis. Using a customary chemical synthesis method for a peptide of formula (1) or (1′), such as a “solid phase synthesis”, single amino acid residues or specific groups as defined in the present specification can be successively coupled.

As used herein, the term “solid phase synthesis” or “solid phase peptide synthesis” refers to a method, well-known to one of ordinary skill in the art, in which a growing peptide chain is linked to a solid support. Solid phase synthesis typically comprises the steps of: (i) covalently binding a first amino acid (whose amino-group is blocked or “protected”) to a solid phase carrier or substrate; (ii) removing the protecting group from the amino-group using a deprotecting agent; (iii) activating the carboxyl of a second amino acid (whose amino-group is blocked) and contacting the second amino acid with the first amino acid bound to the solid phase carrier or substrate so that a dipeptide (whose amino-group is blocked) is obtained; (iv) repeating the peptide bond formation steps and thus the peptide chain is extended from C-terminal to N-terminal; and (v) removing the protecting group of the amino-group and separating the peptide chain from the solid phase carrier with a cleavage agent to yield a peptide.

In one embodiment of the invention, the peptide aldehydes of formula (1) or (1′) according to any of the embodiments described herein, are synthetized using standard solid-phase peptide synthesis protocols employing Fmoc-protection strategy and, for example, a resin as support that is suitable for the synthesis of peptide aldehydes such as an aminomethyl resin bonded with an N-Fmoc-N-methoxy linker (Weinreb AM resin).

Typically, cleavage of the Fmoc protecting group is achieved using a deprotecting agent such as piperidine in dimethylformamide (DMF).

According to one aspect of the invention there is provided a method of solid phase synthesis of a peptide aldehyde of formula (1) or (1′) according to any of the embodiments described in the present specification, comprising the steps of:
a) providing a solid-phase resin substrate;
b) coupling a first amino acid residue to said resin substrate in the presence of a coupling agent, the amino group of said first amino acid being protected by a protecting group;
c) deprotecting said first amino acid residue coupled to said resin substrate;
d) coupling successive amino acid residues to said first amino acid residue in the presence of a coupling agent according to the sequence required, wherein the amino group of said successive amino acids is protected by a protecting group and the deprotection of each successive amino acid residue is performed after each coupling step, the peptide as it is assembled being attached to said resin substrate;
e) detaching the peptide as assembled in step d) from said resin substrate to give the peptide aldehyde of formula (1) or (1′).

Both natural or non-naturally occurring amino acid residues can be employed in the above method and can be assembled in any desired order to prepare the selected peptide aldehyde of formula (1) or (1′).

It is understood that specific reagents different than amino acid residues according to any of the embodiments disclosed in the present specification may also be used in any of the methods herein described when the sequence of the selected peptide aldehyde of formula (1) or (1′) so requires.

) The above method optionally comprises, where necessary, protection of sidechain substituents of the component amino acids, with deprotection being carried out as a final step, either before or after removal of the peptide aldehyde of formula (1) or (1′) from said resin substrate (i.e. before or after step d) of the above method).

In one aspect of the invention, in step b) of the above method the carboxyl group of the selected first amino acid residue is bound to said resin substrate while the amino group of said amino acid is protected, followed by removal of the protecting group, the succeeding steps being as set out above. According to one preferred embodiment, protecting groups are removed using a cleavage mixture containing 5% trifluoroacetic acid (TFA), 1% Triisopropylsilane (TIS) and 94% dichloromethane (DCM) for 30 min.

According to one embodiment, in the above method, an Automated Microwave Peptide Synthesizer is used for the synthesis of the peptide aldehydes of formula (1) or (1′) according to any of the embodiments herein disclosed.

Suitable resin substrates that can be employed in any of the methods according to the present invention are all those resin substrates or supports known in the art, which are specifically suitable for the synthesis of peptide aldehydes.

Preferably, in said step a) of the above method, said solid-phase resin substrate is an aminomethyl resin bonded with an N-Fmoc-N-methoxy linker (Weinreb AM resin).

This specific support is useful for the production of peptide aldehydes and other carboxaldehydes. Following removal of the Fmoc group, for example with 20% piperidine in DMF, acylation of the resin-bound methoxylamine is best effected using DIPCDI/HOAt or HATU/DIPEA activation. This results in formation of a supported Weinreb-type amide which can be reduced to an aldehyde with LiAlH₄.

The abbreviation “DIPCDI” as used herein, refers to N, N′-Diisopropylcarbodiimide, a carbodiimide used in peptide synthesis, while the abbreviation “HOAt” refers to 1-Hydroxy-7-azabenzotriazole, a triazole used as a peptide coupling reagent.

The abbreviation “HATU” as used herein, refers to the compound 1-[Bis(dimethylamino)methylene]-1H-1,2,3-triazolo[4,5-b]pyridinium 3-oxide hexafluorophosphate, also termed as Hexafluorophosphate Azabenzotriazole Tetramethyl Uronium, a reagent used in peptide coupling chemistry to generate an active ester from a carboxylic acid.

As mentioned above, “HATU” is commonly used with Hunig's base (N,N-diisopropylethylamine, abbreviated as “DIPEA”), or triethylamine to form amide bonds. Typically, dimethylformamide (DMF) is used as solvent, although other polar aprotic solvents can also be used.

In cases when Weinreb AM resin is used in step a) of the method of the invention, said step a) further comprises an Fmoc-deprotection step using a 30% piperidine solution in DMF at room temperature (RT).

According to one preferred aspect of the invention, step b) of the method according to any of the embodiments disclosed herein, is carried out using as coupling reagent 2-(1H-benzotriazole-1-yl)-1,1,3,3-tetramethyluronium hexafluorophosphate (HBTU), 2-(1H-7-azabenzotriazol-1-yl)-1,1,3,3-tetramethyluroniumhexafluorophosphate (HATU), and N, N-Diisopropylethylamine DIEA in N-methyl-2-pyrrolidone (NMP). Optionally, the solvent can be filtered off and the coupling procedure can be repeated.

In one embodiment, the method according to the present invention can include one or more washing steps with one or more solvents after each coupling step. Preferably, said one or more washing steps can be performed using dichloromethane (DCM, 3×), N,N-dimethylformamide (DMF, 3×), and DCM (3×).

In one embodiment, in said step d) of the method of the invention according to any of the embodiments described herein, the condensation of said successive amino acid residues is carried out using as coupling agent HBTU, 1-Hydroxy-7-azabenzotriazole (HOAt), and DIEA in NMP.

All couplings steps can be performed for 10 min at 75° C. (2×) and 2×45 min at RT for histidine and cysteine couplings to avoid the epimerization.

In one embodiment of the above method, a chloranil test can be performed after each coupling step to ensure proper coupling.

According to one aspect of the invention, in the method according to any of the embodiments disclosed herein, said step e) is carried out using LiAlH₄.

According to a preferred aspect of the invention, in the method according to any of the embodiments disclosed herein, said step e) further comprises the following steps:
e-i) washing the peptide as assembled in step d) with dry THF and cooling to 0° C.;
e-ii) adding LiAlH₄ and stirring the obtained mixture for 2.25 h;
e-iii) cooling the mixture obtained in the preceding step to 0° C. and diluting it with ethyl acetate;
e-iv) quenching the mixture obtained in step e-iii) with saturated Rochelle's salt solution and stirring for 15 min to ensure quenching;
e-v) filtering the mixture obtained in step e-iv) to remove any solid particulates.
e-vi) extracting the filtrate obtained in step e-v) using ethyl acetate and concentrating the combined ethyl acetate fractions in vacuo to yield said peptide aldehyde of formula (1) or (1′).

The method for the synthesis of a peptide aldehyde of formula (1) or (1′) according to any of the embodiments described in the present specification can be followed by purification of said peptide using any suitable purification technique known in the art for the purification of peptides.

### Pharmaceutical Compositions

According to another aspect of the invention there is provided a pharmaceutical composition comprising at least one peptide aldehyde of formula (1) or (1′) or a derivative, salt or stereoisomer thereof according to any of the embodiments as disclosed in the present specification, and at and at least one pharmaceutically acceptable carrier and/or excipient. The invention further encompasses such compositions for use as a medicament.

As used herein, a “pharmaceutically acceptable carrier” includes any and all solvents, dispersion media, coatings, antibacterial and antifungal agents, isotonic and absorption delaying agents provided they are physiologically compatible. Pharmaceutically acceptable carriers and/or excipients that are preferably employed for the preparation of a composition of the present invention are those commonly used for the preparation of compositions for intra-nasal administration, inhalation, or oral administration, such as buffers or other suitable excipients known to a person skilled in the art to provide improved transfer, delivery, tolerance and the like. Agents capable of increasing the shelf life of the composition and preserving the effectiveness of the peptide aldehydes of the invention can also be used in the composition and include wetting agents, emulsifiers, preservatives or buffers. The composition may optionally comprise a pharmaceutically acceptable diluent or adjuvant, or preservatives to maintain microbiological stability in liquid formulations, such as benzalkonium chloride or the like.

The choice of the pharmaceutical carrier, excipient, diluent or adjuvant can be selected with regard to the intended route of administration and standard pharmaceutical practice. The pharmaceutical compositions herein disclosed may be preferably for human use.

In one embodiment, the pharmaceutical composition of the invention comprises at least one peptide aldehyde of formula (1) or (1′), or a derivative, salt or stereoisomer thereof according to any of the embodiments as disclosed in the present specification, in a therapeutically effective amount. Preferably, said composition comprises an amount of at least one peptide aldehyde of formula (1) or (1′), or derivative, salt or stereoisomer thereof, sufficient to exert a protective, preventive and/or therapeutic effect against a viral infection, particularly a Coronavirus infection such as a SARS-CoV-2 infection.

In one embodiment, the peptide, or salt or stereoisomer thereof is present in the composition in an amount ranging from about 0.005 mg to about 100 mg.

In one embodiment, the peptide, or salt or stereoisomer thereof is present in the composition in an amount ranging from about 0.1 mg to about 2 mg, or from about 0.25 mg to about 1 mg, or from about 0.5 mg to about 1 mg, or from about 0.25 to about 0.75 mg. Exemplary unit doses include about 0.1 mg, about 0.2 mg, about 0.25 mg, about 0.3 mg, about 0.4 mg, about 0.5 mg, about 0.6 mg, about 0.7 mg, about 0.75 mg, about 0.8 mg, about 0.9 mg, about 1 mg, about 1.25 mg, about 1.5 mg, about 1.75 mg, or about 2 mg.

In one aspect, the pharmaceutical composition comprising at least one peptide aldehyde of formula (1) or (1′), or derivative, salt or stereoisomer thereof according to any of the embodiments of the present invention, comprises at least the 20%, 30%, 40%50%, 60%, 70%, 80%, 90%, 98%, 99% of said peptide aldehyde of formula (1) or (1′).

In one aspect of the present invention, the pharmaceutical composition according to any of the embodiments herein disclosed can be in any form provided it is suitable for intra-nasal administration or inhalation, aerosol or nebulization, or oral administration. The dispersion of a pharmaceutical composition according to any of the embodiments of the present invention in the form of droplets facilitates its delivery through the respiratory tract, increasing the bioavailability of the peptide aldehydes of the invention at the level of the mucosal membranes.

A suitable pharmaceutical composition according to the present invention may be in a form selected from solution, nasal drops, nasal aerosol and/or spray, nebulized solution or suspension, powder, microspheres, pill, capsule, tablet or beads. Any of the foregoing formulations may be appropriate in treatments and therapies in accordance with the present invention, provided that the active agent(s) in the formulation is(are) not inactivated by the formulation and the formulation is physiologically compatible.

According to one embodiment, said peptide aldehydes or compositions described herein are administered to a subject by contacting the mucosal tissues of the gastrointestinal tract. For example, said one or more peptide aldehydes or compositions according to any of the embodiments described herein may be formulated for delivery to one or more of the small intestines and large intestine. Preferably, in the case of oral administration, the pharmaceutical compositions according to any of the embodiments of the present invention, are in the form of gastro-resistant microspheres, capsules, pills or tablets. As used herein, the term “gastro-resistant” is interchangeable with the term “enteric” and is referred to the property of such microspheres, capsules, pills or tablets to cross the gastric tract without being damaged and then release the active ingredient (i.e. the peptide aldehyde of the invention) within the intestine. As an example, said microspheres, capsules, pills or tablets may be coated by a gastro-resistant or enteric coating, the solubility of the coating being dependent on the pH, in particular in such a manner that it prevents the release of the active ingredient in the stomach but permits the release of the active ingredient at some stage after the pharmaceutical composition has emptied from the stomach. Gastro-resistant or enteric coatings can comprise at least one polymer being insoluble in aqueous solutions having pH value of less than 4.5 and at least one further excipient selected from plasticizers, anti-tacking agents, pigments and/or surface-active substances.

It hence forms part of the present invention also an oral composition comprising a peptide, or salt or stereoisomer thereof or a composition according to any one of the embodiments described herein in the form of gastro-resistant microspheres.

In various embodiments, the pharmaceutical compositions of the invention may be formulated to have a delayed-release profile, i.e. not immediately release the active ingredient(s) upon ingestion; rather, postponement of the release of the active ingredient(s) until the composition is lower in the gastrointestinal tract.

All the pharmaceutical compositions described herein may be prepared by employing standard preparation techniques known in the pharmaceutical field. The compositions can also be provided already aliquoted in single dosages or in single dosage fractions.

The pharmaceutical compositions of the invention can also be packaged either in multi-dose or in single-dose containers.

Non-limiting examples of devices that can be used for aerosol administration of a pharmaceutical composition according to the invention include a nebulizer (or a small volume nebulizer, SVN), a metered-dose inhaler (or pressurized metered-dose inhaler, pMDI), or a dry powder inhaler (DPI).

Nebulizers use compressed gasses (air, oxygen, and nitrogen) or ultrasonic or mechanical power to break up the pharmaceutical composition into small aerosol droplets that can be directly inhaled into the mouth or nose. The smaller particles and slow speed of the nebulized aerosol are advocated to increase penetration to the target sites in the middle and superior meatuses and the paranasal sinuses.

According to one embodiment, the peptide aldehydes of formula (1) or (1′) or the pharmaceutical compositions according to any of the embodiments herein disclosed can be administered to a subject in need thereof in the form of an aerosol composition using a nebulizer operated by a suitable propellant, such as air or oxygen, by a compressor, or by an electric power device. The selection of the most suitable dispensing device for the administration of a composition according to any of the embodiments of the invention can be made on the basis of many different factors, such as the type of patient, easiness of use, or frequency of administration.

Another object of the present invention is related to a nasal spray device comprising a peptide, or salt or stereoisomer thereof or a composition according to any one of the embodiments described in the present specification.

According to an embodiment of the invention, said nasal spray device comprises a bottle and/or vial containing a dose of the pharmaceutical composition to be administered and a spray pump or piston system connected to a terminal tip or syringe, for example in the form of a nozzle, to allow the delivery of an optimal volume of the composition directly into the nasal or oronasal cavity. The dose can be metered by the spray pump or could have been premetered during manufacture. A nasal spray unit can be designed for unit dosing or can discharge up to several hundred metered sprays of formulation containing one or more peptide aldehydes of formula (1) or (1′) according to the invention.

### Therapy

The invention further encompasses the peptide aldehyde of formula (1) or (1′), or derivative, salt or stereoisomer thereof, or the pharmaceutical composition according to any of the embodiments herein discloses for use as a medicament, in particular for use in the prevention and/or treatment of viral infections and/or of one or more diseases associated to or caused by viral infections.

As used herein, the term “viral infections” refers to an abnormal state or condition characterized by viral invasion of a health cell, uses of the cell's reproductive machinery to multiply or replicate and ultimately lyse the cell resulting in cell death, release of viral particles and the infection of other cells by the newly produced progeny viruses. As used herein, the expression “for the treatment and/or prevention of viral infections” means for use to inhibit the replication of a particular virus, to inhibit viral transmission, or to prevent the virus from establishing itself in its host, and/or to ameliorate or alleviate the symptoms of the disease caused or associated to the viral infection. The term “prevention” may hence also encompass the treatment of a subject who is not affected by a viral infection but is believed to be at risk of viral infection.

Viral infections for which treatment or prevention with the compounds or compositions of this invention will be particularly useful include infections of the respiratory tract.

According to a preferred embodiment of the invention, said viral infection is hence a viral infection of the respiratory tract, particularly a Coronavirus infection, and even more preferably a SARS-COV-2 infection.

The peptide aldehydes of formula (1) or (1′), or derivative, salt or stereoisomer thereof, or the pharmaceutical compositions according to any of the embodiments herein disclosed are particularly indicated for the treatment of SARS-COV-2 infections in an initial stage, or for the treatment of subjects presenting mild symptoms and/or asymptomatic subjects.

In one preferred embodiment, the peptide aldehyde of formula (1) or (1′), or derivative, salt or stereoisomer thereof, or the pharmaceutical composition according to any of the embodiments herein disclosed is for use as inhibitor of the main protease (M^{pro}) of a Coronavirus, and particularly of SARS-COV-2. According to one aspect of the invention, the peptide aldehyde of formula (1) or (1′), or derivative, salt or stereoisomer thereof, or the pharmaceutical composition according to any of the embodiments herein disclosed can be used as inhibitor of the activity of said main protease in vivo.

Non limiting examples of diseases associated to or caused by viral infections that can benefit from the present invention include interstitial pneumonia, acute lung injury (ALI), and acute respiratory distress syndrome (ARDS).

The invention further encompasses also the peptide aldehyde or the pharmaceutical composition according to any of the embodiments herein discloses for use in a method of treatment of a subject in need thereof comprising a step of administering to said subject at least one peptide aldehyde or pharmaceutical composition of the invention in a therapeutically effective amount. As previously mentioned, a “therapeutically effective amount” of the peptide aldehyde or pharmaceutical composition of the invention employed for the treatment and/or prevention of viral infections according to the present invention, may vary depending upon many different factors. These include means of administration, the age, weight and physiological state of the subject being treated, and other medicaments administered. Thus, treatment dosages could be varied to optimize safety and efficacy.

According to particular embodiment of the invention, the expression “therapeutically effective amount” refers to an amount of a peptide aldehyde of formula (1) or (1′), or derivative, salt or stereoisomer thereof according to the present invention, which is effective, upon single or multiple dose administration to the patient, in controlling the growth, replication or infectivity of a virus, or in decreasing the amount of virus. As used herein, “controlling the growth” of the virus refers to slowing, interrupting, arresting or stopping the viral transformation of cells or the replication and proliferation of the virus and does not necessarily indicate a total elimination of the virus.

The peptides of the invention may be administered singly or in combination with each other or other antiviral agents. Typically, the peptide aldehydes of formula (1) or (1′) according to any of the embodiments of the invention can be administered preferably once or twice daily. However, other amounts, including substantially lower or higher amounts may also be administered.

In accordance with certain embodiments, the peptide aldehydes of formula (1) or (1′) or pharmaceutical compositions of the invention can be administered one or more times daily to subjects in need thereof, such as children and/or adults. For example, the peptide aldehyde of formula (1) or (1′) or the pharmaceutical compositions according to any of the embodiments of the present invention can be administered once daily, two times daily, or about three times daily or more. In certain embodiments, the regimen of the peptide aldehydes of the compositions of the invention can be administered to subjects in need thereof for a prolonged period.

Typically, treatment with direct administration of the peptides of formula (1) or (1′) or a composition according to any of the embodiments of the present invention can be carried out for a period of time sufficient to reduce, prevent or ameliorate symptoms.

Preferably, the peptide aldehydes of formula (1) or (1′) or the pharmaceutical composition are administered to a subject in need thereof by way of inhalation, intra-nasal administration, nebulization, or aerosol or by any other acceptable routes of administration.

Where allowed the invention encompasses also a medical treatment comprising the administration to subjects in need thereof of a peptide aldehyde or of a composition of the invention in any of the embodiments provided in the present description in a therapeutically effective amount.

Hence disclosed is a method of preventing and/or treating a patient suffering from a viral infection, and particularly a SARS-COV-2 infection, comprising the following step: i. administering to patients in need thereof an effective amount of at least one peptide aldehyde of formula (1) or (1′) or a pharmaceutical composition according to any one of the embodiments described in the present specification.

Preferably said peptide aldehyde will be administered according to a therapeutically effective dose regimen as determined on the basis of several factors such as the severity of the disease, means of administration, age, surface area, weight, sex, and condition of the subject in need thereof.

For example, said peptide aldehyde or a pharmaceutical composition according to any of the embodiments of the invention may be administered as described herein for at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, or at least about 26 weeks. In some embodiments, the said peptide aldehyde or pharmaceutical composition is administered for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In some embodiments, said peptide aldehyde or pharmaceutical composition may be administered for at least about 1 year, at least about 2 years, at least about 3 years, at least about 4 years, or at least about 5 years, for example for about one year.

According to a preferred embodiment, step i. of the above method will be performed by way of intra-nasal administration, more preferably by way of administration with a nasal spray or aerosol.

In one embodiment, the levels of one or more peptide aldehydes of the invention that are delivered to the subject undergoing the above treatment may be monitored by means of a quantitative and/or qualitative analysis performed on a biological sample obtained from said subject.

Having thus described different embodiments of the present invention, it should be noted by those skilled in the art that the disclosures herein are exemplary only and that various other alternatives, adaptations, and modifications may be made within the scope of the present invention. Accordingly, the present invention is not limited to the specific embodiments as illustrated herein.

Examples are reported below which have the purpose of better illustrating the embodiments disclosed in the present description, such examples are in no way to be considered as a limitation of the previous description and the subsequent claims.

### EXAMPLES

### Example 1—Second Generation of AT1001 Analogues

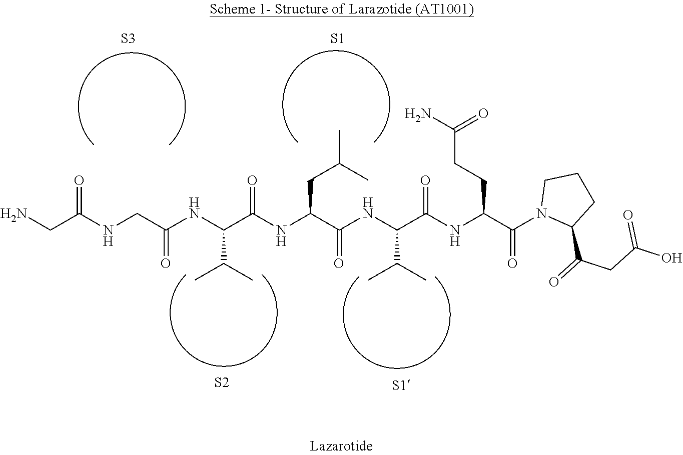

Based on previous results, a second generation of AT1001 analogues was designed, endowed with an aldehydic “warhead” able to establish a reversible covalent bond with Cys145 of the enzyme, increasing the binding affinity vs. the macromolecule. In details, the structural studies revealed that AT1001 positioned the peptide bond between Leu4 and Val5 at ˜3 Å from Cys145, responsible for proteolytic activity of M^{pro}.

Thus, the carboxylic group of Leu4 has been converted to aldehyde, and the octapeptide cut to tripeptide, as molecular dynamics investigations suggested that the residues Gly1, Gln6, Pro7 and Gly8 largely fluctuated over the simulation contributing less to the line-up of ligand-protein complex.

Considering that the side chain of Val3 and Leu4 are respectively accommodated into S2 and S1 pockets, in the new compound collection these sequence positions were preserved, just varying the structural features of the new inserted residues in terms of size, polarity, donor/acceptor H-bond groups. Moreover, the Gly2 was maintained or substituted with non-standard amino acids, without affecting the H-bonds with Glu166. A small library of tripeptides that have been screened in silico was generated.

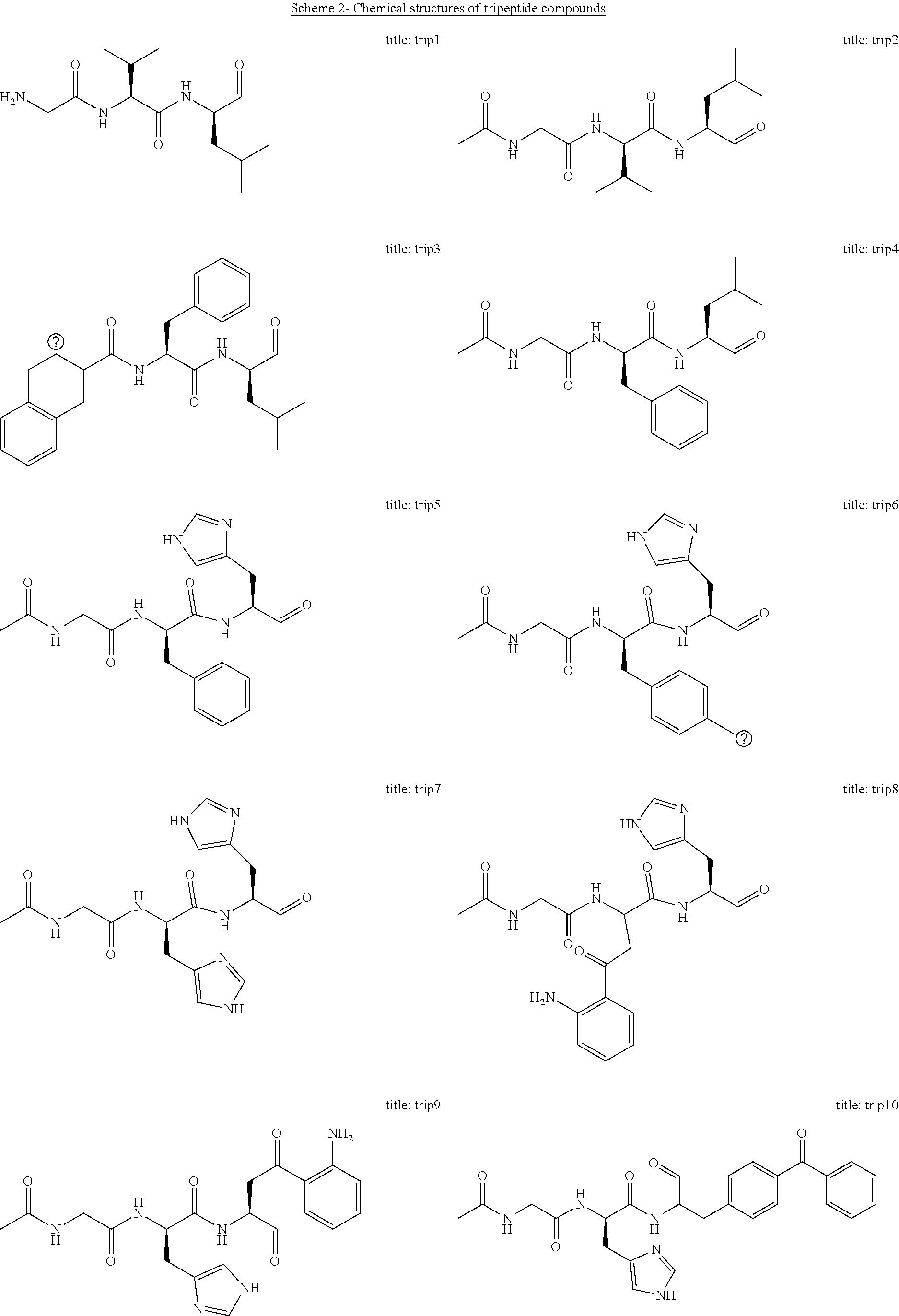

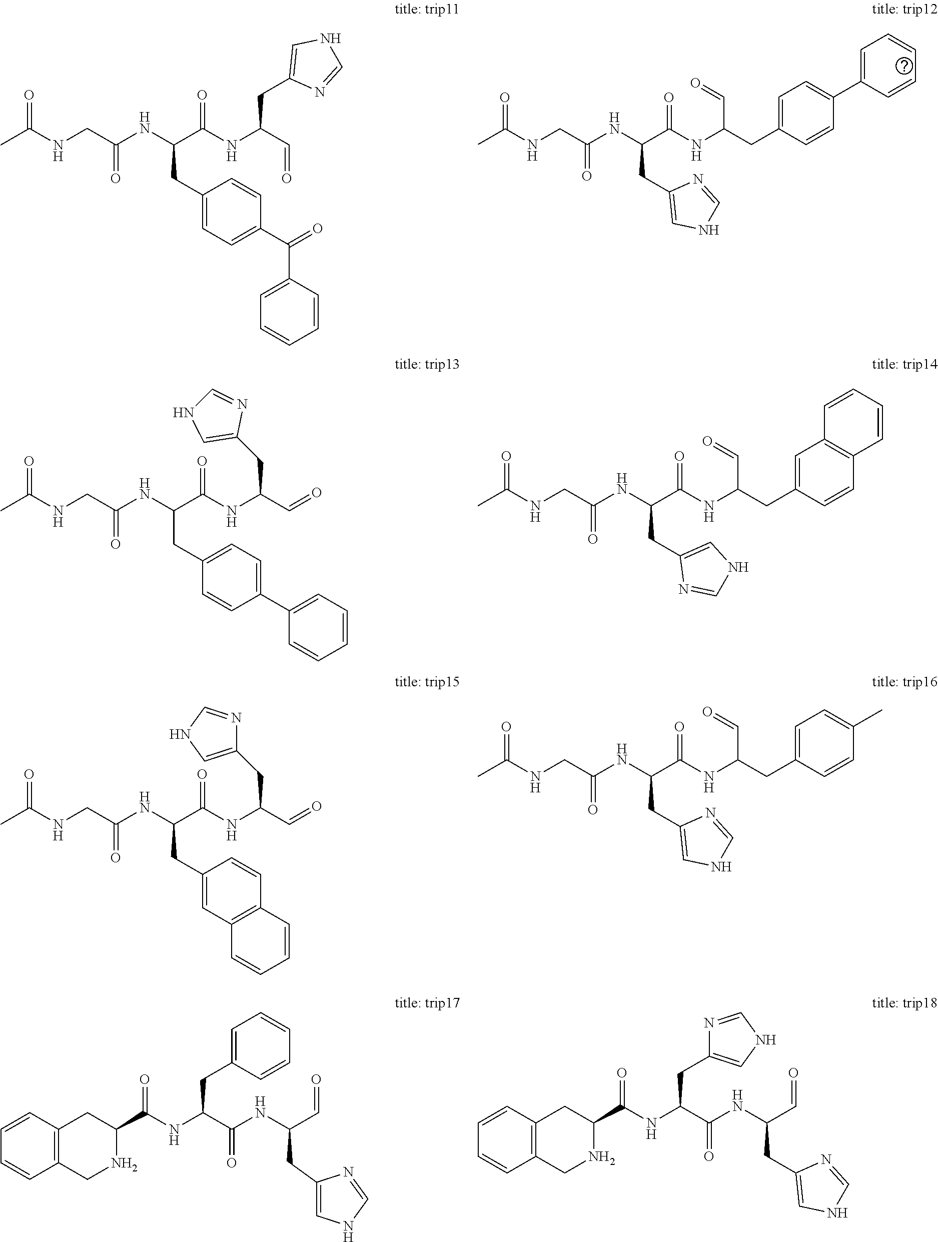

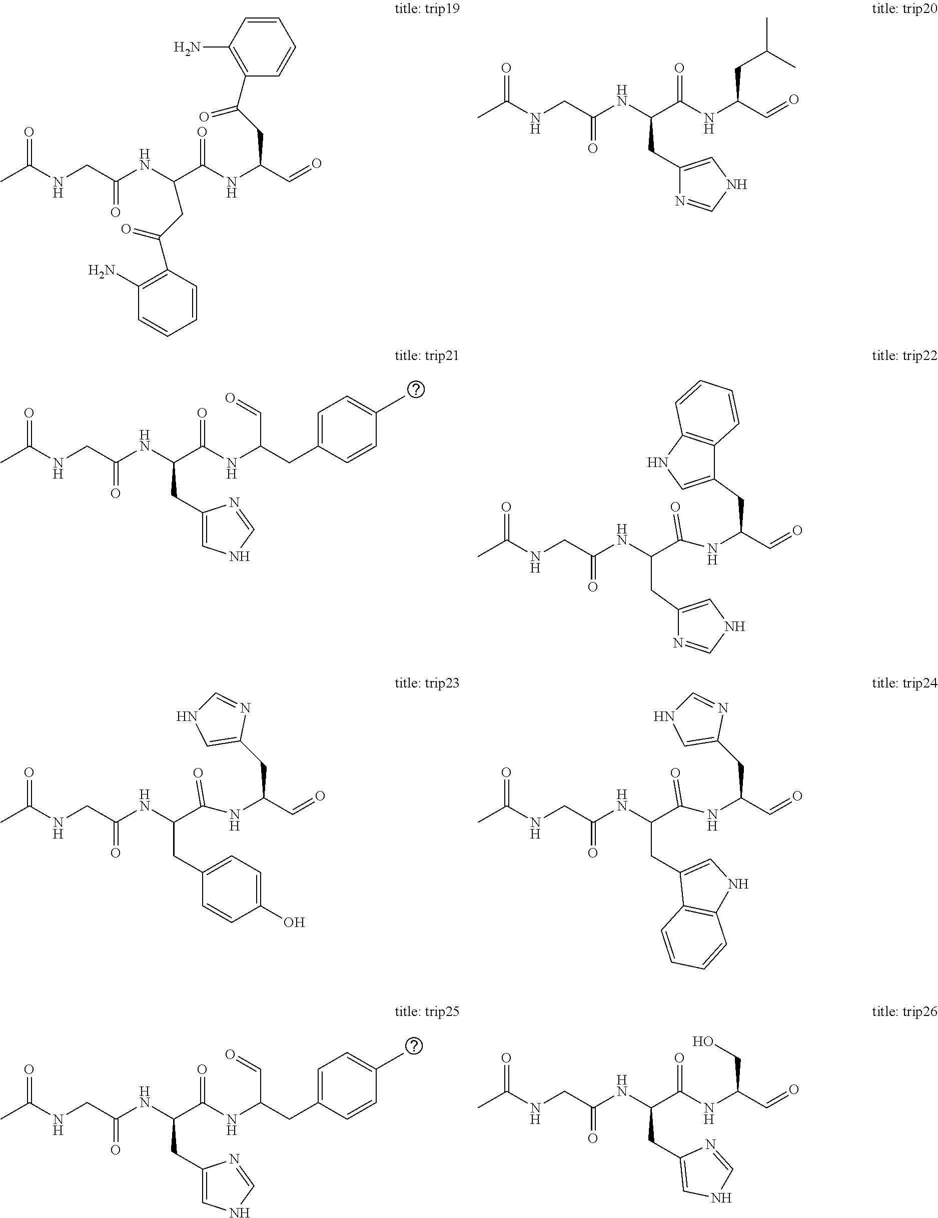

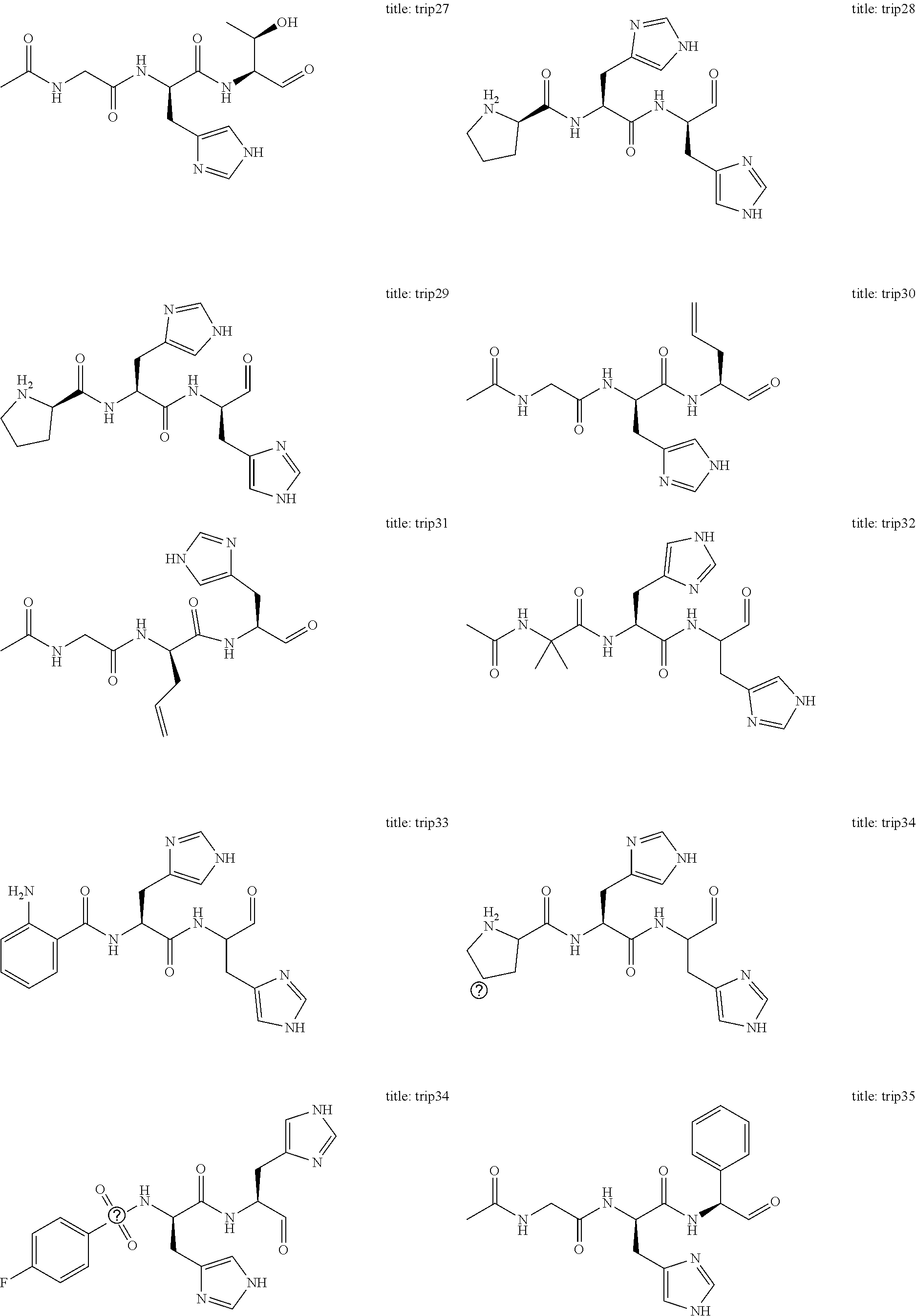

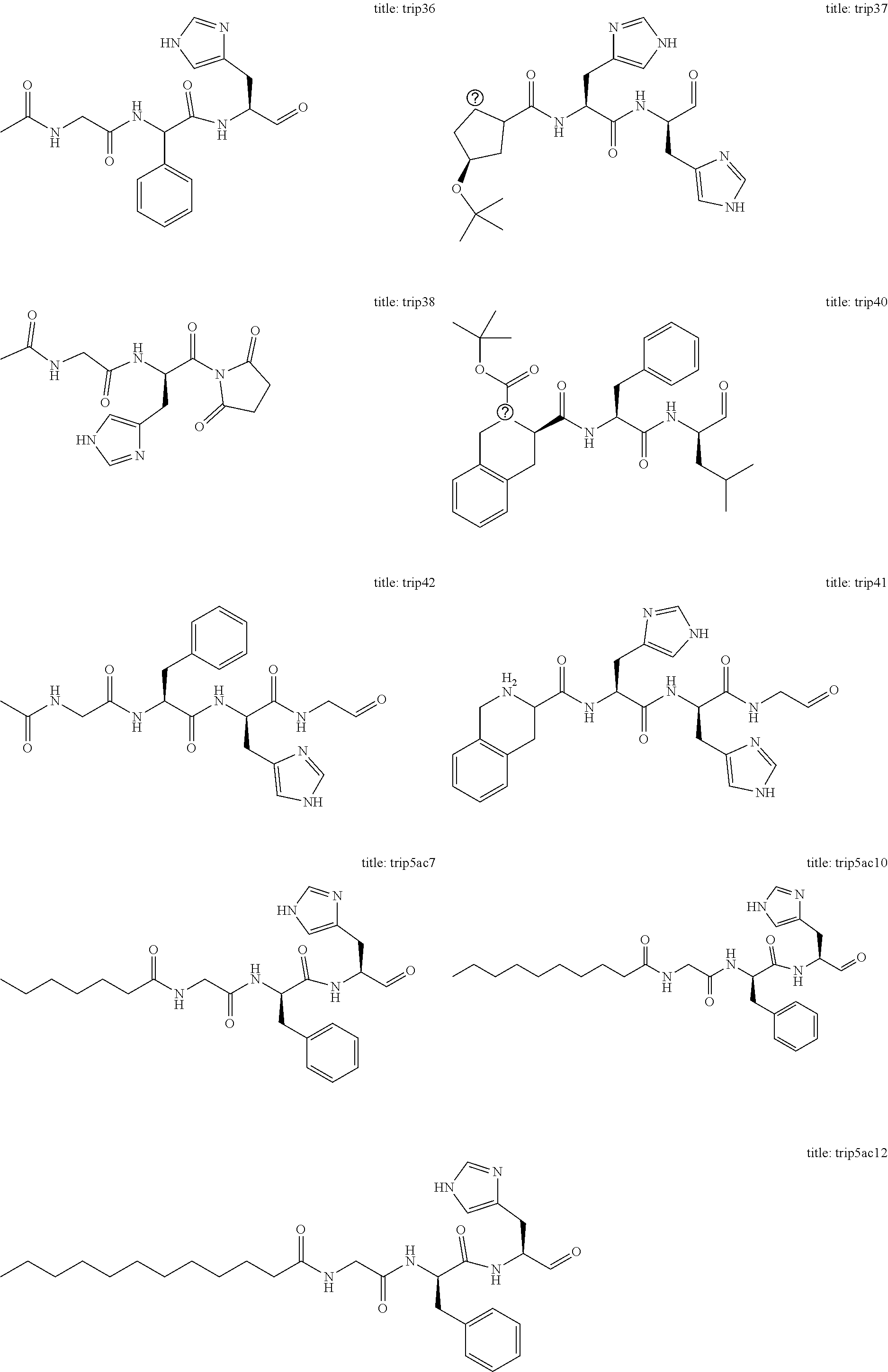

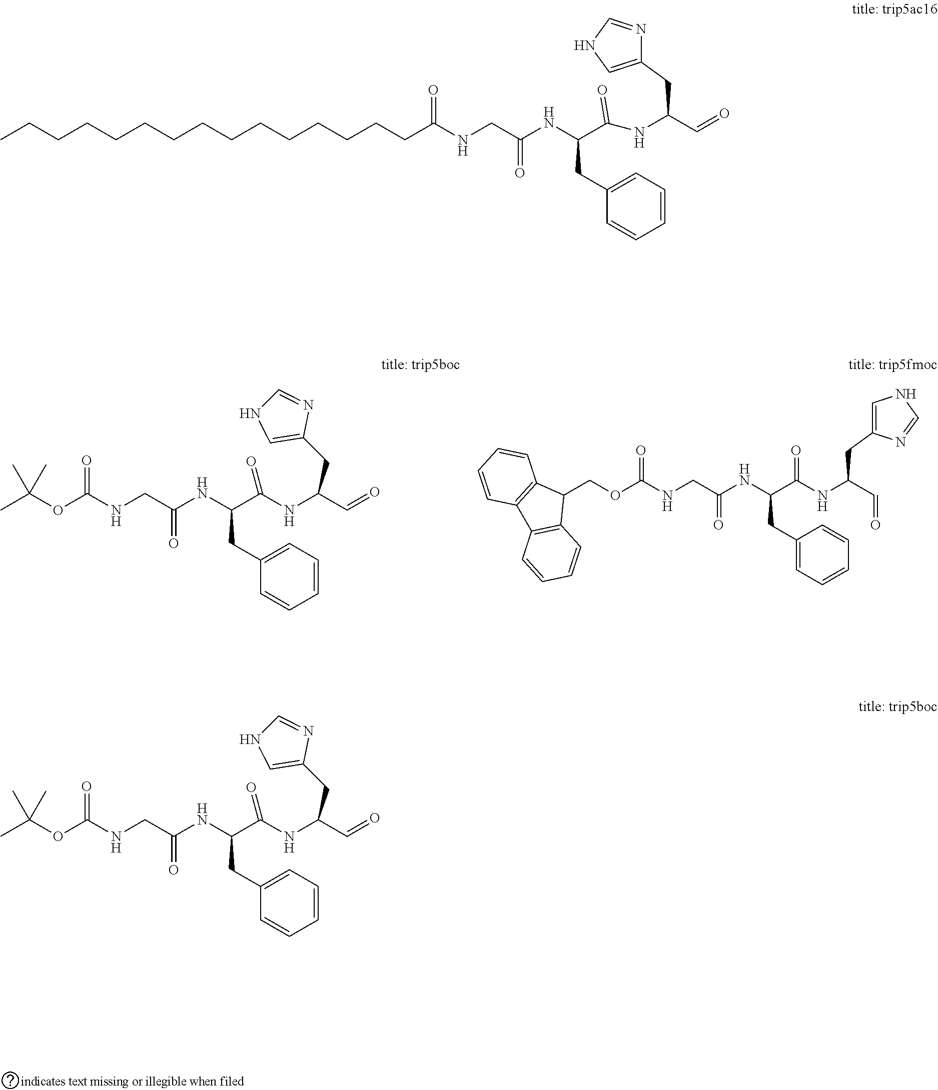 [SMILES: CCCCCCCCCCCCCCCC(=O)NCC(=O)N[C@H](Cc1ccccc1)C(=O)N[C@H](C=O)Cc1c[nH]cn1 ; CC(C)(C)OC(=O)NCC(=O)N[C@H](Cc1ccccc1)C(=O)N[C@H](C=O)Cc1c[nH]cn1 ; O=C[C@H](Cc1c[nH]cn1)NC(=O)[C@@H](Cc1ccccc1)NC(=O)CNC(=O)OCC1c2ccccc2-c2ccccc21 ; CC(C)(C)OC(=O)NCC(=O)N[C@H](Cc1ccccc1)C(=O)N[C@H](C=O)Cc1c[nH]cn1]

Eight compounds were filtered in silico to get a minimum of structure-activity relationship. Among these peptides, two tetrapeptides (TRIP41 and 42) presenting the aldehyde group on a glycine were also included to experimentally prove the optimal length of shorter analogues for warhead positioning. The selected analogues were synthesized as follow.

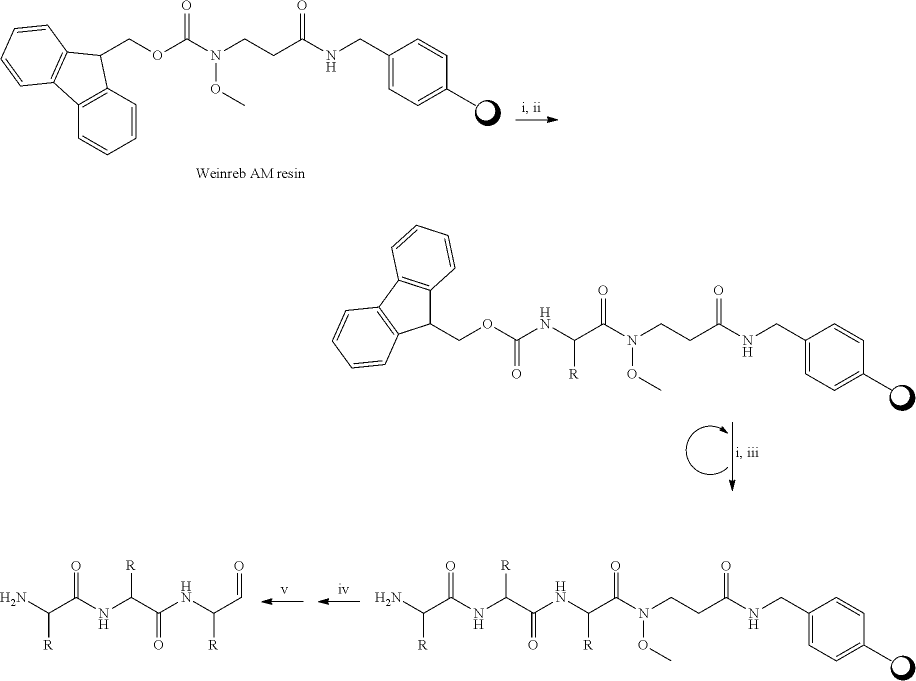

Scheme 3—Reagents and conditions: (i) 30% piperidine/DMF, 1×3 min, 1×10 min; (ii) Fmoc-AA(Pg)—OH (3 eq), HATU (3 eq), Dipea (6 eq) 2×10 min, MW 75° C.; (iii) Fmoc-AA(pg)-OH (3 eq), HBTU (3 eq), HOAt (6 eq), 2×10 min, MW 75°; (iv) LiAH4 (5 eq), THF (0.05 M resin), 0° C. 2.25 h; (v) CH₂Cl₂//TES/TFA.

The putative binding of the selected compounds towards the M^{pro} was evaluated by FRET assay, using the calpeptin as positive control.

TABLE 1

| Compound ID | IC50 [μM] |
|---|---|
| TRIP 3 | >4.25E+02 |
| TRIP 5 | 2.51E+00 |
| TRIP 7 | >2.13E+02 |
| TRIP 18 | 3.78E+01 |
| TRIP 21 | >4.25E+02 |
| TRIP 23 | 1.98E+02 |
| TRIP 37 | >1.70E+02 |
| TRIP 41 | >4.25E+02 |
| TRIP 42 | >4.25E+02 |
| Calpeptin | 2.43E+00 |

The experimental data showed that three compounds presented an inhibitory activity of M^{pro} spanning into micromolar range. It is noteworthy that TRIP5 presented an IC₅₀=2.51±0.24 μM comparable to the calpeptin (IC₅₀=2.41+0.20 μM). Moreover, TRIP5 showed a K_{D}=1.26±0.12 μM similar to calpeptin value (K_{D}=1.22+0.10 μM) suggesting that reversible covalent bond formation. Collectively, the structure-activity relationships suggested that is crucial the positioning of a hydrophobic group in S2 cavity and small-sized moiety to get H-bonds with Glu166 next to S3 pocket. It should be highlighted that these structural observations agree with SAR of reported peptide and peptidomimetic inhibitors of M^{pro}. As the endogenous substrate of M^{pro} accommodates a glutamine residue in S1 pocket, different proposed inhibitors are endowed with a γ-lactame mimicking this amino acid side chain. The results observed for TRIP5 showed that it is possible substitute the glutamine with a histidine, as it gives H-bonds with His163 and Glu166 and its structural rigidity reduce the loss of entropy after enzyme binding. Overall, the obtained outcomes lay foundation for the design of new tripeptide generation.

The putative antiviral activity was evaluated by cell-based assay on Vero cells infected by SARS-Cov-2, along with SA (RG) and UK (NVDBB) variants. The cellular assays were carried out on TRIP5 and its two derivatives, TRIP5boc and TRIP5ac7, to enhance the cell membrane permeability of TRIP5. The experimental outcomes showed the antiviral activity of tested compounds against SAR-COV-2 and two variants, revealing that improving the membrane crossing gives rise to a better biological activity.

TABLE 2
Activity of compounds received from Prof. M. Sala against SARS-COV-2 in Vero cells.

| | Virus inoculum | | | | | | | | | |
|---|---|---|---|---|---|---|---|---|---|---|
| | | | | | Antiviral | | | | | |
| | Antiviral | | Antiviral | | activity EC₅₀^{a} | | | | | |
| | activity EC₅₀^{a} | | activity EC₅₀^{a} | | UK (NVDBB) strain | | | | | |
| | 1074 # 2 strain | | SA (RG) strain | | Vero cells | | Cytotoxicity | | | |
| | Vero cells | | Vero cells | | 6 μl/10 ml | | Cell morphology | | Cell growth | |
| Compound | 3 μl/10 ml | | 10 μl/10 ml | | 8 μl/10 ml | | (MCC)^{b} | | (CC₅₀)^{c} | |
| Remdesivir | Not available | | 0.84 | μM | Not available | | 80 | μM | ND | |
| | 1.01 | μM | 1.43 | μM | 0.19 | μM | ≥80 | μM | >80 | μM |
| | 1.17 | μM | 1.56 | μM | 0.19 | μM | 80 | μM | 59.09 | μM |
| T-705 | Not available | | 52.31 | μg/ml | Not available | | >200 | μg/ml | ND | |
| | 76.15 | μg/ml | >200 | μg/ml | 23.39 | μg/ml | >200 | μg/ml | >200 | μg/ml |
| | 81.79 | μg/ml | 89.44 | μg/ml | 23.39 | μg/ml | >200 | μg/ml | >200 | μg/ml |
| TRIP 5 | Not available | | >100 | μM | Not available | | 100 | μM | ND | |
| | 63.14 | μM | >100 | μM | 35.91 | μM | >100 | μM | >100 | μM |
| | 68.98 | μM | >100 | μM | 44.72 | μM | ≥100 | μM | >100 | μM |
| TRIP 43 | Not available | | 54.69 | μM | Not available | | 100 | μM | ND | |
| | 44.72 | μM | >100 | μM | 46.66 | μM | >100 | μM | >100 | μM |
| | 55.70 | μM | >100 | μM | 39.86 | μM | ≥100 | μM | >100 | μM |
| TRIP 44 | Not available | | 26.15 | μM | Not available | | >100 | μM | ND | |
| | 3.46 | μM | 48.90 | μM | 8.41 | μM | ≥100 | μM | >100 | μM |
| | 6.56 | μM | 44.72 | μM | 2.01 | μM | >100 | μM | >100 | μM |

^{a}Effective concentration required to reduce virus plaque formation by 50%. Virus input was 100 plaque forming units (PFU).
^{b}Minimum cytotoxic concentration that causes a microscopically detectable alteration of cell morphology.
^{c}Cytotoxic concentration required to reduce cell growth by 50%.
^{d}Not determined.

The three compounds were also tested on two unrelated viruses, cytomegalovirus and varicella-zoster virus, to verify the selectivity against SARS-Cov-2. The biological assays demonstrated no activity against cytomegalovirus and varicella-zoster virus.

TABLE 3
Activity of compounds received from Prof. M. Sala on 31, Mar. 2021
against cytomegalovirus (HCMV) in human embryonic lung (HEL) cells

| | Antiviral activity EC₅₀ (μM)^{a} | | Cytotoxicity (μM) | |
|---|---|---|---|---|
| Compound | AD-169 strain | Davis strain | Cell morphology (MCC)^{b} | Cell growth (CC₅₀)^{c} |
| TRIP 5 | >20 | >100 | 100 | ND |
| | >20 | >100 | 100 | ND |
| TRIP 43 | >20 | >100 | 100 | ND |
| | >20 | >100 | 100 | ND |
| TRIP 44 | >20 | >100 | 100 | ND |
| | >20 | >100 | 100 | ND |
| Ganciclovir (GCV) | 5.00 | 2.01 | 394 | ND |
| | 6.54 | 1.65 | ≥394 | ND |
| Cidofovir (CDV) | 1.39 | 0.92 | 317 | ND |
| | 1.08 | 0.25 | 317 | ND |

^{a}Effective concentration required to reduce virus plaque formation by 50%. Virus input was 100 plaque forming units (PFU).
^{b}Minimum cytotoxic concentration that causes a microscopically detectable alteration of cell morphology.
^{c}Cytotoxic concentration required to reduce cell growth by 50%.
^{d}Not determined.

TABLE 4
Activity of compounds received from Prof. M. Sala on 31, Mar. 2021 against
varicella-zoster virus (VZV) in human embryonic lung (HEL) cells

| | Antiviral activity EC₅₀ (μM)^{a} | | Cytotoxicity (μM) | |
|---|---|---|---|---|
| | TK⁺ VZV strain | TK⁻ VZV strain | Cell morphology | Cell growth |
| Compound | OKA | 07-1. | (MCC)^{b} | (CC₅₀)^{c} |
| TRIP 5 | >100 | >100 | >100 | ND |
| | >100 | >100 | >100 | ND |
| TRIP 43 | >100 | >100 | >100 | ND |
| | >100 | >100 | >100 | ND |
| TRIP 44 | >100 | >100 | >100 | ND |
| | >100 | >100 | >100 | ND |
| Aciclovir (ACV) | 43.16 | 139.59 | >444 | ND |
| | 14.65 | 84.63 | >444 | ND |
| Brivudine (BVDU) | 0.27 | 5.89 | >300.3 | ND |
| | 0.17 | 4.53 | >300.3 | ND |

^{a}Effective concentration required to reduce virus plaque formation by 50%. Virus input was 20 plaque forming units (PFU).
^{b}Minimum cytotoxic concentration that causes a microscopically detectable alteration of cell morphology.
^{c}Cytotoxic concentration required to reduce cell growth by 50%.
^{d}Not determined.

### Material and Methods

### Molecular Docking

CovDock was used for covalent docking calculation for designed compounds against M^{pro}. The Pose Prediction (Thorough) docking mode was applied. A cutoff of 2.5 kcal/mol was used to retain poses for further refinement, with a maximum of 999 poses to keep for this step. Output poses per ligand reaction site was site to 100.

### General Procedure for Solid Phase Peptide Synthesis (SPPS) Using the Weinreb Aminomethyl (AM) Resin

The tripeptides were synthesized using an Automated Microwave Peptide Synthesizer from Biotage AB (Initiator+Alstra). Peptides were synthesized on a Weinreb AM resin (0.150 g, loading 0.5 mmol/g) previously Fmoc-deprotected by a 30% piperidine solution in DMF (1×3 min and 1×10 min) at room temperature (RT). A Chloranil test was then applied. After a positive Chloranil test (colored beads), the first amino acid, Na-Fmoc-Xaa-OH, was linked on to the resin, using as coupling reagent 2-(1H-benzotriazole-1-yl)-1,1,3,3-tetramethyluronium hexafluorophosphate (HBTU) (3 equiv.), 2-(1H-7-azabenzotriazol-1-yl)-1,1,3,3-tetramethyluroniumhexafluorophosphate (HATU) (3 equiv.), and N,N-Diisopropylethylamine DIEA (6 equiv.) in N-methyl-2-pyrrolidone (NMP). The solvent was then filtered off and the procedure was then repeated. Chloranil test was then applied to ensure proper coupling. The peptide resin was washed with dichloromethane (DCM, 3×), N,N-dimethylformamide (DMF, 3×), and DCM (3×) and the Fmoc deprotection protocol, described above, was repeated after each coupling step. The following protected amino acids were then added on to the resin using as coupling reagent HBTU (3 equiv.), 1-Hydroxy-7-azabenzotriazole (HOAt) (3 equiv.), and DIEA (6 equiv.) in NMP.

All couplings were achieved for 10 min at 75° C. (2×) and 2×45 min at RT for histidine and cysteine couplings to avoid the epimerization.

General Procedure for Cleavage from Weinreb AM Resin

The on-resin tripeptide (1.0 equiv.) was swollen in dry THF (0.05 M resin) and cooled to 0° C. LiAlH₄ (5.0 equiv.) was added portion wise and the mixture allowed to stir for 2.25 h. The mixture was again cooled to 0° C. and diluted with ethyl acetate (5 mL). The mixture was then quenched with saturated Rochelle's salt solution (5 mL) and allowed to stir for 15 min to ensure quenching. The mixture was then filtered using a fritted filter to remove any solid particulates. The resulting filtrate was extracted three times using ethyl acetate. The combined ethyl acetate fractions were concentrated in vacuo to yield the desired tripeptide.

Finally, protecting group were removed using a cleavage mixture containing 5% TFA, 1% Triisopropylsilane (TIS) and 94% DCM for 30 min.

### Purification and Characterization

All crude peptides were purified by RP-HPLC on a preparative C18-bonded silica column (Phenomenex Kinetex Biphenyl 100 Å, 100×21.2 mm, 5 μm) using a Shimadzu SPD 20 A UV/VIS detector, with detection at 214 and 254 nm. Mobile phase was: (A) H₂O and (B) ACN, both acidified with 0.1% TFA (v/v). Injection volume was 5000 μL; flow rate was set to 17 mL/min. The following gradient was employed: 0-18 min, 1-40% B, 18.01-20 min, 40-70% B, 20.01-21 min, 70-90% B, 21.01-23 min, returning to 1% B. Analytical purity and retention time (tr) of each peptide were determined using HPLC conditions in the above solvent system (solvents A and B) programmed at a flow rate of 0.600 mL/min, fitted with C-18 column Phenomenex, Kinetex Biphenyl 100 Å C18 column (100×3.00 mm, 2.6 μm). LC gradient was the following: 0-7 min, 1-40% B, 7.01-8 min, 40-90% B, 8.01-9 min, returning to 1% B, 9-11 min, isocratic for 2 min. All analogues showed >97% purity when monitored at 220 nm. Homogeneous fractions, as established using analytical HPLC, were pooled and lyophilized.

Ultra-high-resolution mass spectra were obtained by positive ESI infusion on a LTQ Orbitrap XL mass spectrometer (Thermo Scientific, Germany), equipped with the Xcalibur software for processing the data acquired. The sample was dissolved in a mixture of water and methanol (50/50) and injected directly into the electrospray source, using a syringe pump, at constant flow (15 μL/min).

### FRET Assay for TRIP

Target enzyme: SARS-COV-2 M^{pro} (3264-3569aa); Proteros, WTH_20_1
Target enzyme conc: 20 nM
Probe and conc.: Dabcyl-KTSAVLQSGFRKM-E(Edans)-NH2: 5 μM
Binding buffer: 20 mM HEPES pH 7.5, 1 mM DTT, 1 mM EDTA, 100 mM NaCl, 0.01% Tween20
   Compounds: 20 mM, 25 mM and 50 mM Compound in 100% DMSO
   Assay plates: 384 well U bottom, PP, black, low volume (Corning, 4514)
   Eppendorf® LoBind microcentrifuge tubes (Z666491, Sigma-Aldrich)
   Reaction volume: 10 μL
   Reaction temperature: Room temperature

### General Assay Protocol

1) Add appropriate volume of target enzyme in reaction buffer to cpd wells and C+ wells
2) Add appropriated volume of binding buffer to C-wells
3) Add 85 nL compound in 100% DMSO
4) Incubate for 15 min at room temperature
5) Add appropriate volume of substrate in reaction buffer
6) Measure reporter displacement after 30 min

### Compound Stocks (100% DMSO)

| | |
|---|---|
| 50 mM | TRIP 3 |
| 50 mM | TRIP 5 |
| 25 mM | TRIP 7 |
| 50 mM | TRIP 18 |
| 50 mM | TRIP 21 |
| 50 mM | TRIP 23 |
| 20 mM | TRIP 37 |
| 50 mM | TRIP 41 |
| 50 mM | TRIP 42 |
| 50 mM | Calpeptin |

1. A peptide aldehyde of the formula (1′):
R₃—R₂—R₁—H
or a derivative, salt or stereoisomer thereof wherein:
H is hydrogen;
R₁ is a single amino acid residue of valine, histidine, substituted alanine selected from 3-anthraniloyl-alanine and 2-naphtylalanine, phenylalanine, substituted phenylalanine selected from meta-hydroxyphenylalanine, p-bromophenylalanine, para-benzoylphenylalanine (Bpa), para-biphenylalanine and p-chlorophenylalanine, tyrosine, tryptophan, serine, threonine, asparagine, glutamine, or substituted glycine selected from cyclohexylglycine, allylglycine and α-Phenylglycine;
R₂ is a single amino acid residue of valine, serine, histidine, substituted alanine selected from 3-anthraniloyl-alanine and 2-naphtylalanine, phenylalanine or substituted phenylalanine selected from para-benzoylphenylalanine (Bpa), para-biphenylalanine and p-chlorophenylalanine, tyrosine, threonine, tryptophan, or substituted glycine selected from cyclohexyl glycine, allylglycine and α-Phenylglycine;
R₃ is a single amino acid residue of glycine, substituted glycine selected from cyclohexyl glycine and phenylglycine, valine, 3-methylvaline, proline or substituted proline selected from thioproline and trans-4-tert-Butoxy-L-proline, leucine, isoleucine, alanine, (α-methyl)alanine, or is a group selected from N-(1,2,3,4-Tetrahydro-3-isoquinolinylcarbonyl) group, N-(2-Aminobenzoyl) group, and [(4-Fluorophenyl)sulfonyl] group;
wherein said R₃ single amino acid residue optionally comprises an N-terminal protecting group; and
wherein said derivative of the peptide aldehyde of formula (1′) is a peptide of formula (1′) in which the R₁—H group is replaced by a succinimide group, or is a peptide of formula (1′) in which the aldehyde group is replaced by a reactive “warhead” group selected from: ketone; Michael acceptor, α-ketoamide; nitrile; bisulfite; electrophilic heterocycles; boronic acids; cyanamide; isothiocyanate; S-electrophile; electron-deficient (hetero)arene; vinyl sulfone and vinyl sulphonamide; acrylamide; propiolamide; N-methyl isoxazolium; fumaric acid ester; allenamide; propionitrile; alkenyl-/alkynyl-substituted heteroarene; haloalkane; α-halomethyl amide/ester/ketone; epoxide; aziridine; nitroalkane; α-cyanoacrylamide; α-cyanoenone; electron-deficient (hetero)arene with leaving group; acyloxymethylketone; hydroxymethyl and alkoxymethylketone; nonactivated terminal alkynes; or is a peptide of formula R₂—R₁—H (1″) wherein R₁ is histidine and R₂ is phenylalanine comprising 1H-Pyrrol-2-carbonyl as N-terminal protecting group.

2. A peptide aldehyde of the formula (1′) according to claim 1, or a derivative, salt or stereoisomer thereof wherein:
H is hydrogen;
R₁ is a single amino acid residue of valine, histidine, substituted alanine selected from 3-anthraniloyl-alanine and 2-naphtylalanine, phenylalanine, substituted phenylalanine selected from para-benzoylphenylalanine (Bpa), meta-hydroxyphenylalanine, p-bromophenylalanine, para-biphenylalanine and p-chlorophenylalanine, tyrosine, tryptophan, serine, threonine, asparagine, glutamine, or substituted glycine selected from cyclohexylglycine allylglycine and α-Phenylglycine;
R₂ is a single amino acid residue of valine, serine, histidine, substituted alanine selected from 3-anthraniloyl-alanine and 2-naphtylalanine, phenylalanine or substituted phenylalanine selected from para-benzoylphenylalanine (Bpa), para-biphenylalanine and p-chlorophenylalanine, tyrosine, threonine, tryptophan, or substituted glycine selected from cyclohexyl glycine, allylglycine and α-Phenylglycine;
R₃ is a single amino acid residue of glycine, substituted glycine selected from cyclohexyl glycine and phenylglycine, valine, 3-methylvaline, proline or substituted proline selected from thioproline and trans-4-tert-Butoxy-L-proline, leucine, isoleucine, alanine, (α-methyl)alanine, or is a group selected from N-(1,2,3,4-Tetrahydro-3-isoquinolinylcarbonyl) group, N-(2-Aminobenzoyl) group, and [(4-Fluorophenyl)sulfonyl] group;
wherein said R₃ single amino acid residue optionally comprises an N-terminal protecting group; and
wherein said derivative of the peptide aldehyde of formula (1′) is a peptide of formula (1′) in which the R₁—H group is replaced by a succinimide group; or is a peptide of formula R₂—R₁—H (1″) wherein R₁ is histidine and R₂ is phenylalanine comprising 1H-pyrrol-2-carbonyl as N-terminal protecting group.

3. The peptide aldehyde according to claim 1, wherein said N-terminal protecting group is selected from C₂₋₁₆ acyl, 1-(1H-indol-2-yl)carbonyl, 1-(1H-indol-5-yl)carbonyl, 1H-pyrrol-3-carbonyl, 1H-pyrrol-2-carbonyl, 2-furancarbonyl, 2-(4-pyridinyl)-1,3-thiazole-4-carbonyl, cyclopent-3-ene-1-carbonyl, cyclohexanecarbonyl, cyclopentylacetyl, 1-(4-methylphenyl)cyclopropancarbonyl, cyclopropylcarbonyl, cycloesylacetyl, 1,3-thiazolidine-4-carbonyl, t-butyloxycarbonyl or fluorenylmethyloxycarbonyl.

4. The peptide aldehyde according to claim 1, wherein:
R₁ is a single amino acid residue of valine, histidine, or tyrosine;
R₂ is a single amino acid residue of phenylalanine, histidine, or tyrosine;
R₃ is a single amino acid residue of glycine or trans-4-tert-Butoxy-L-proline, or is the N-(1,2,3,4-Tetrahydro-3-isoquinolinylcarbonyl) group, wherein said R₃ single residue optionally comprises an N-terminal protecting group selected from C₂₋₁₆ acyl, t-butyloxycarbonyl or fluorenylmethyloxycarbonyl.

5. The peptide aldehyde according to claim 1, wherein:
R₁ is a single amino acid residue of histidine;
R₂ is a single amino acid residue of phenylalanine, histidine, or tyrosine; and
R₃ is a single amino acid residue of glycine or is the N-(1,2,3,4-Tetrahydro-3-isoquinolinylcarbonyl) group, wherein said R₃ single amino acid residue optionally comprises an N-terminal protecting group selected from C₂₋₁₆ acyl, t-butyloxycarbonyl or fluorenylmethyloxycarbonyl.

6. The peptide aldehyde according to claim 1, wherein:
R₁ is a single amino acid residue of histidine;
R₂ is a single amino acid residue of phenylalanine; and
R₃ is a single amino acid residue of glycine; wherein said R₃ single residue optionally comprises an N-terminal protecting group selected from C₂₋₁₆ acyl, t-butyloxycarbonyl or fluorenylmethyloxycarbonyl.

7. The peptide aldehyde according to claim 1, wherein said peptide or salt or stereoisomer thereof is selected from:

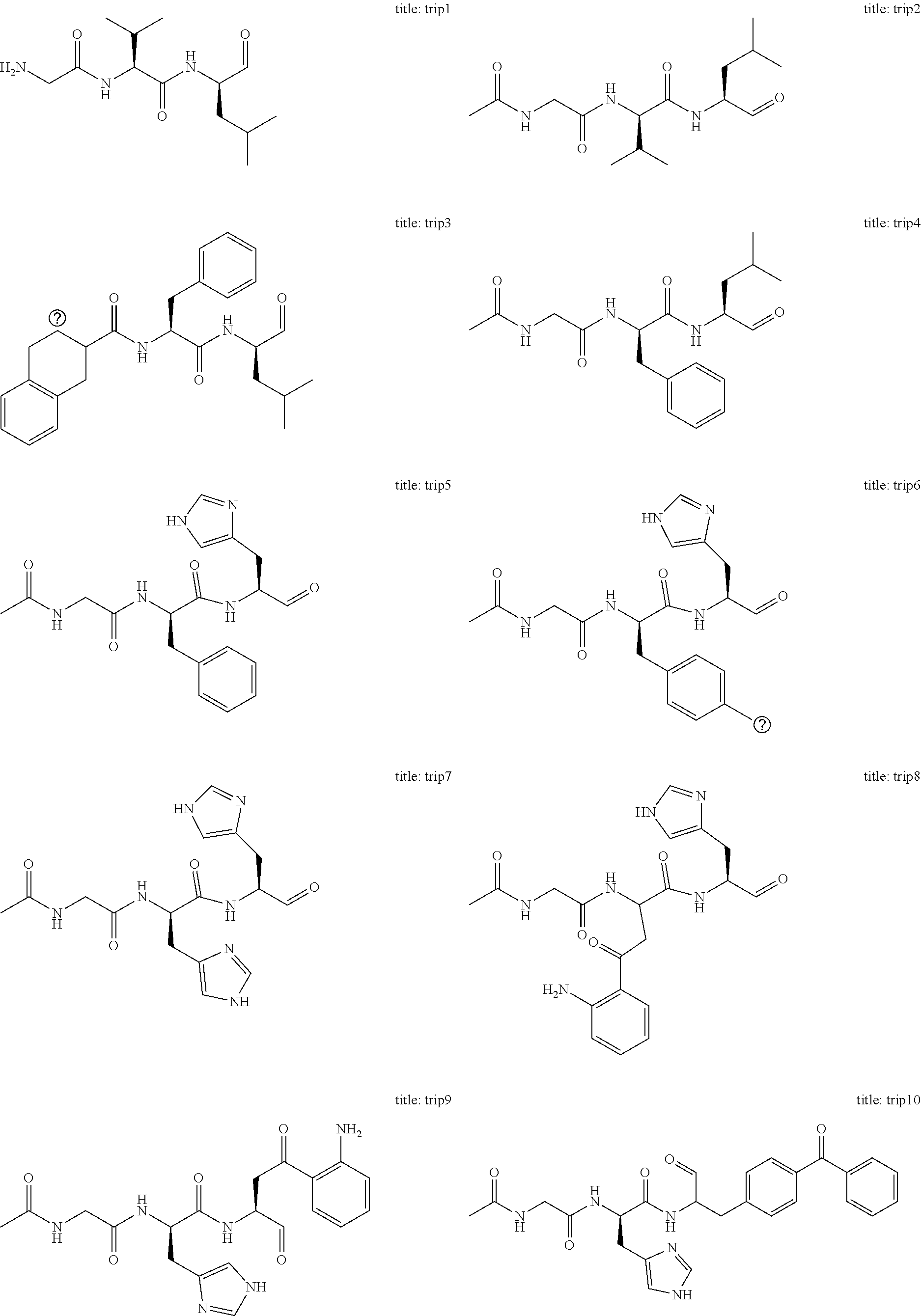

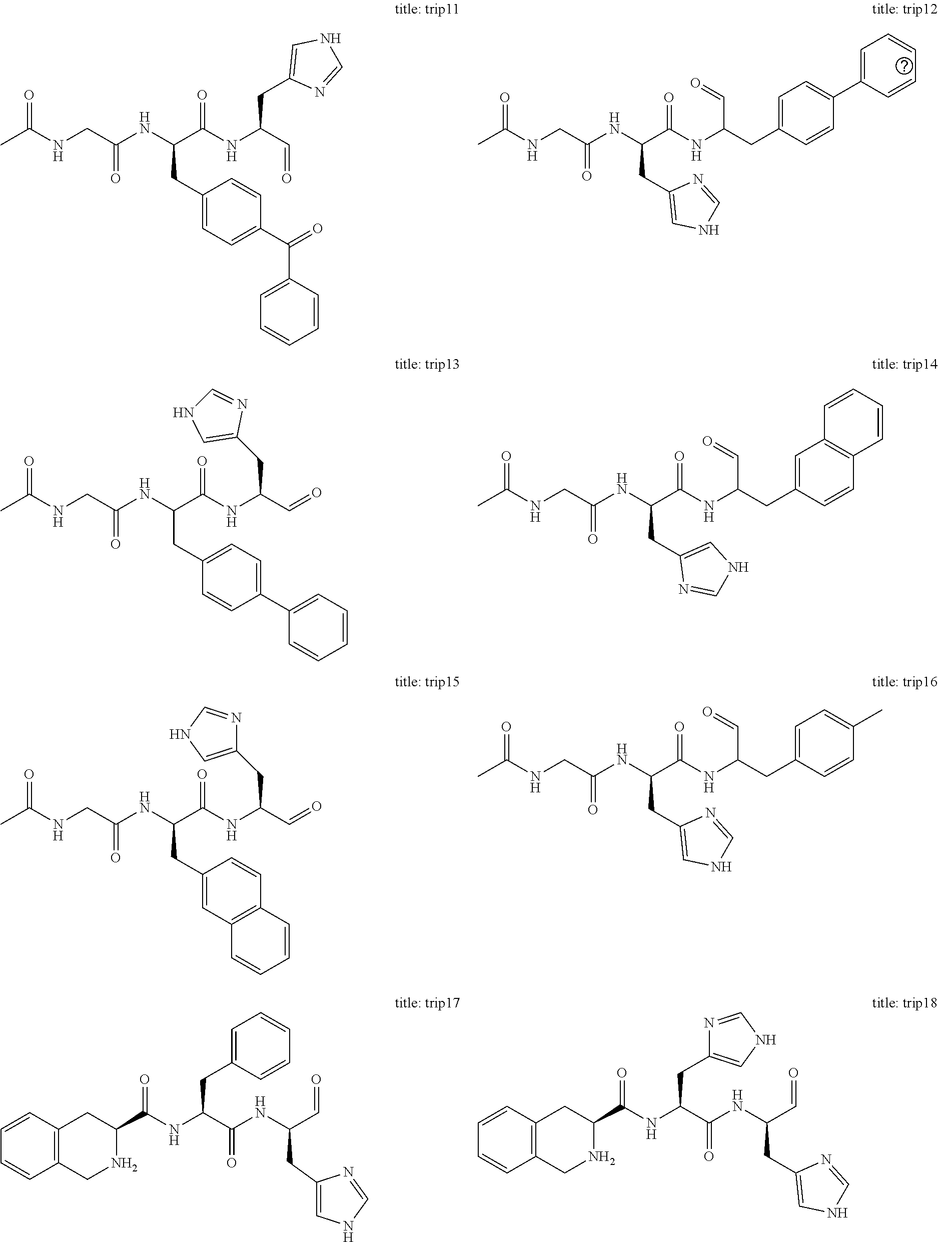

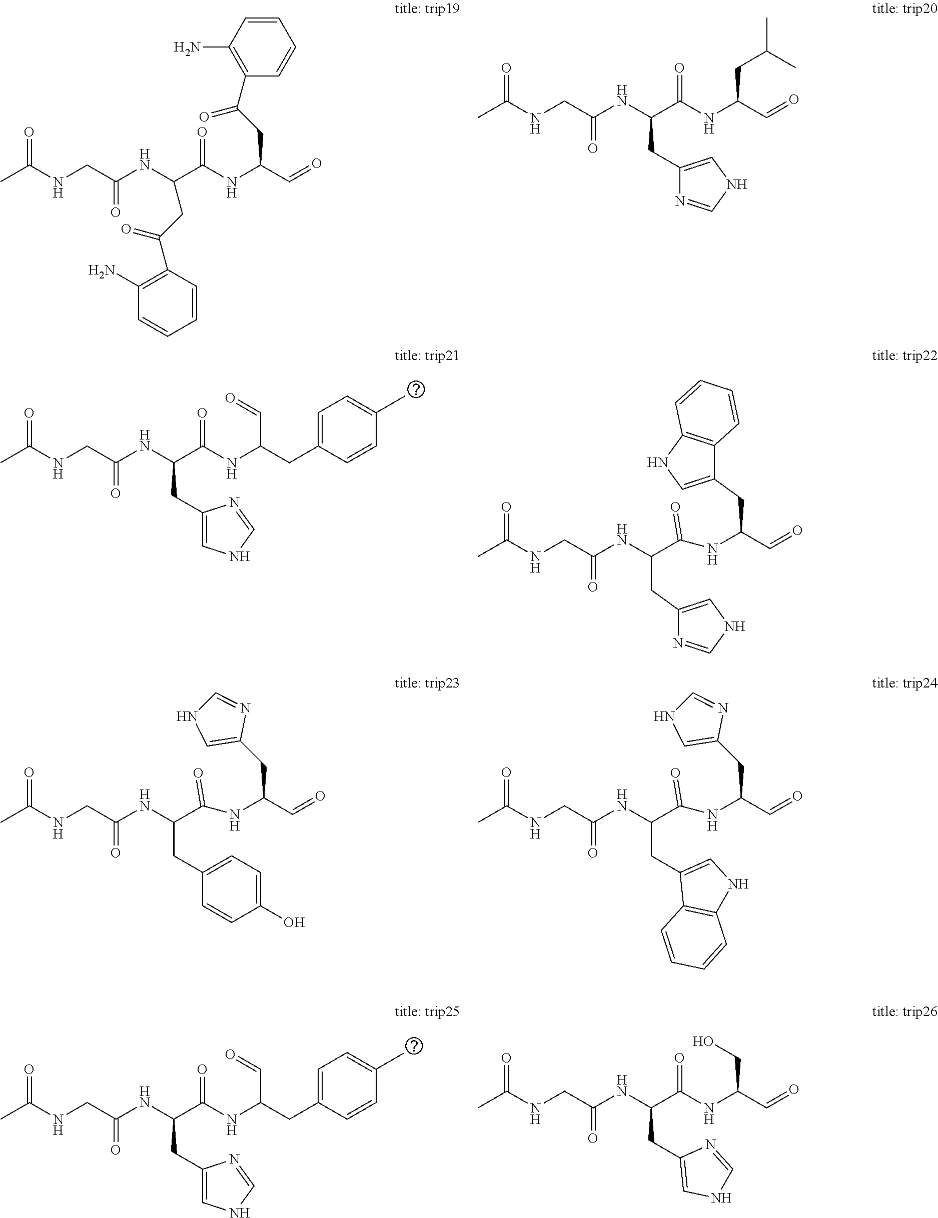

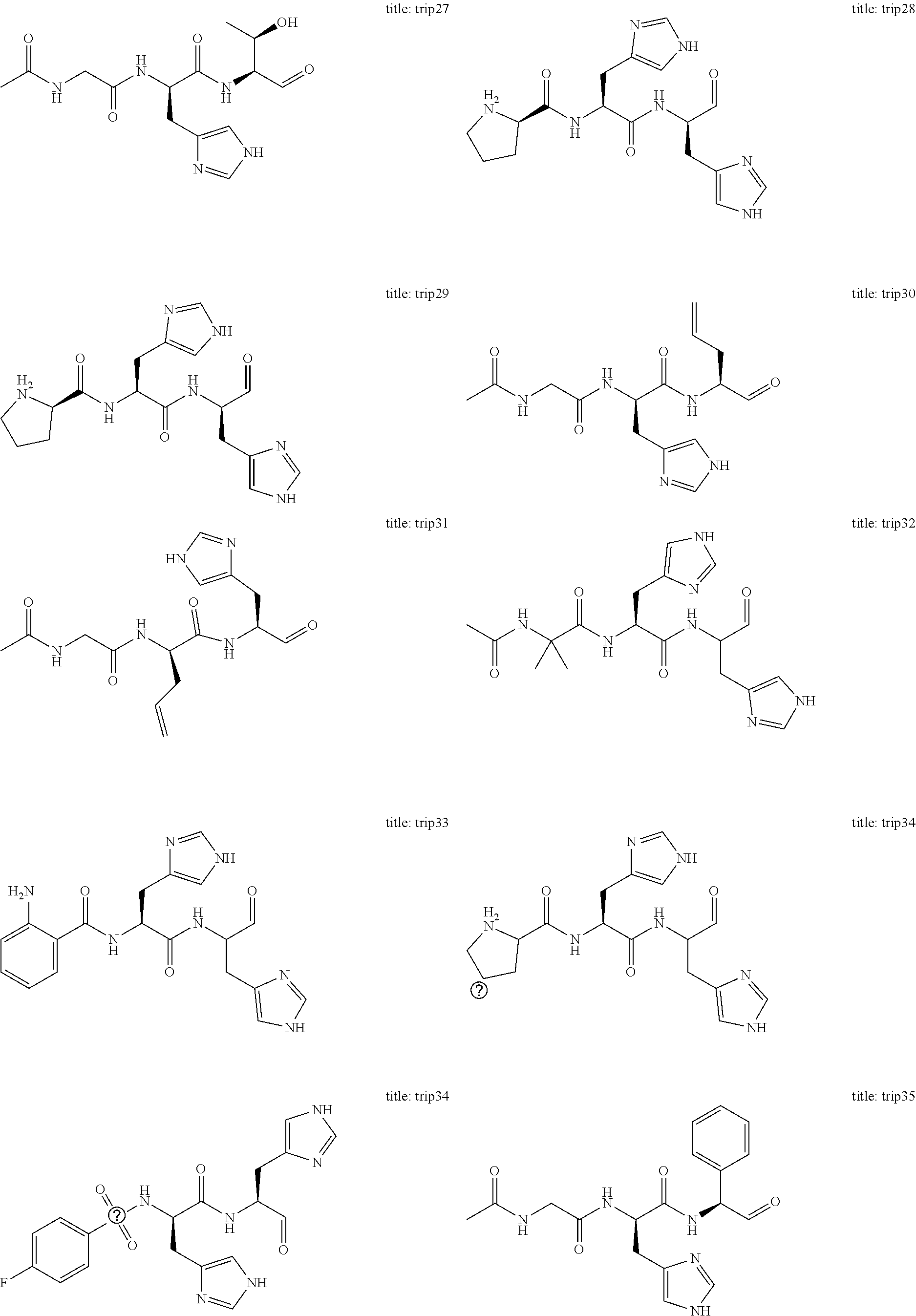

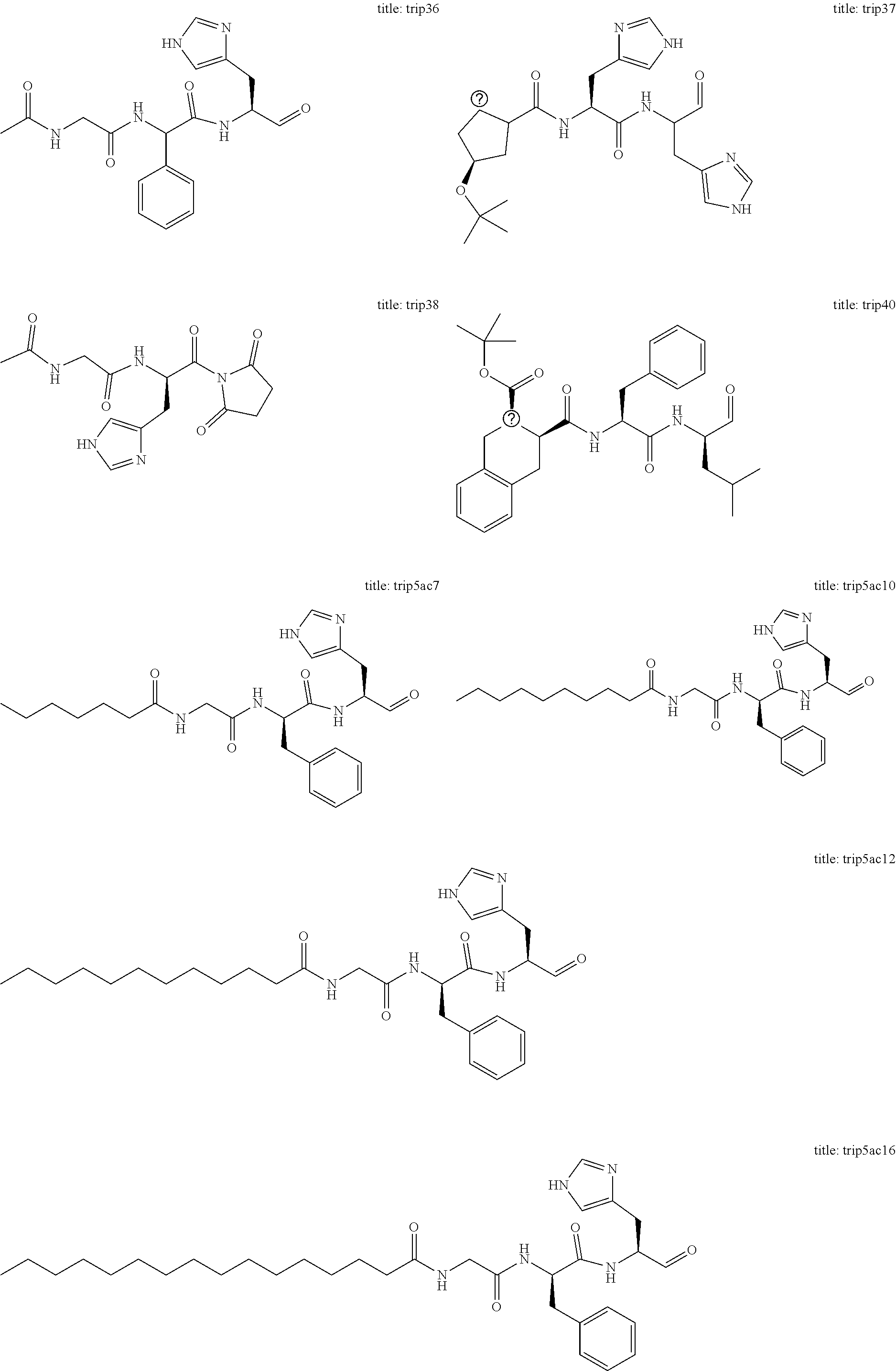

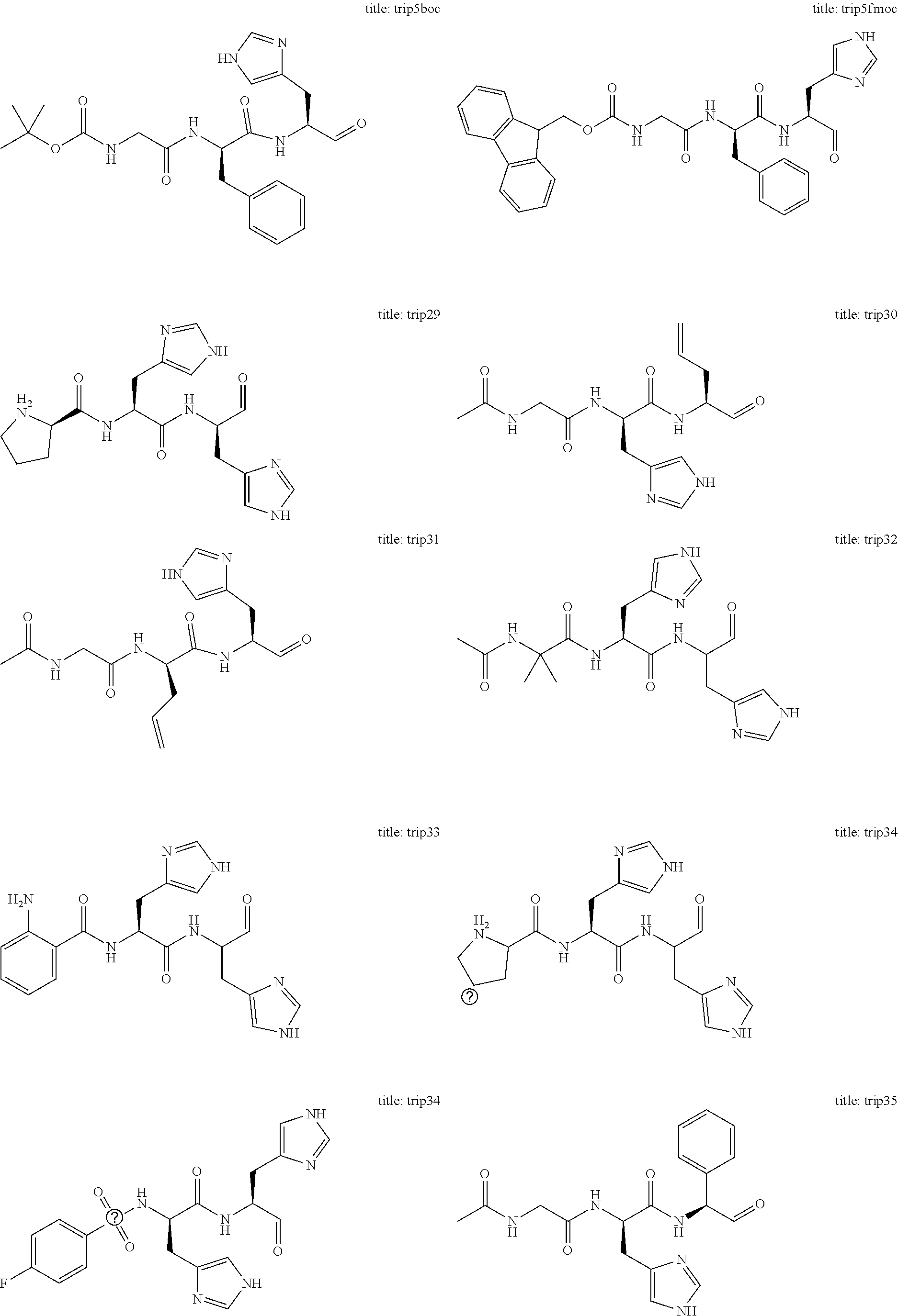

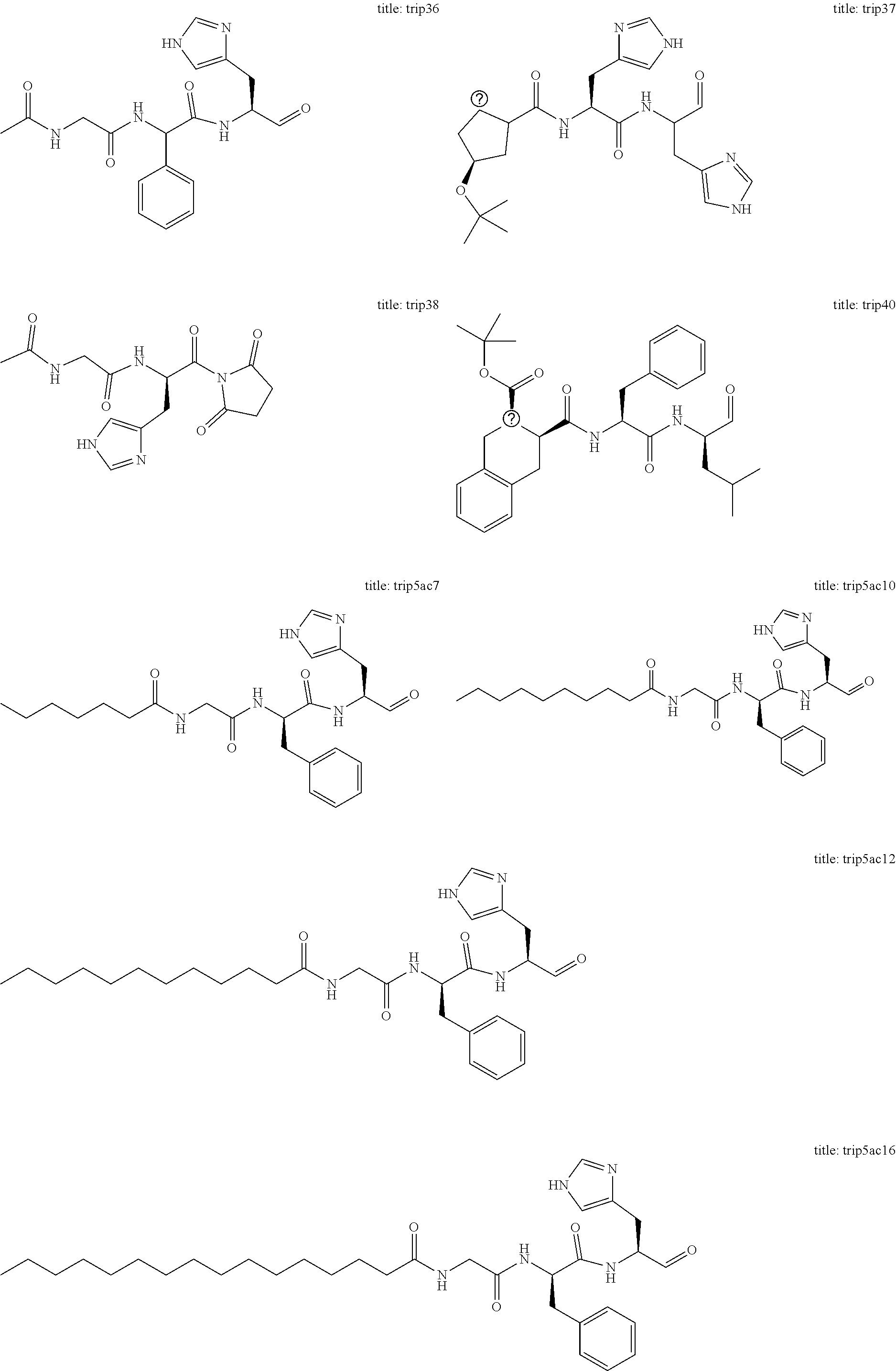

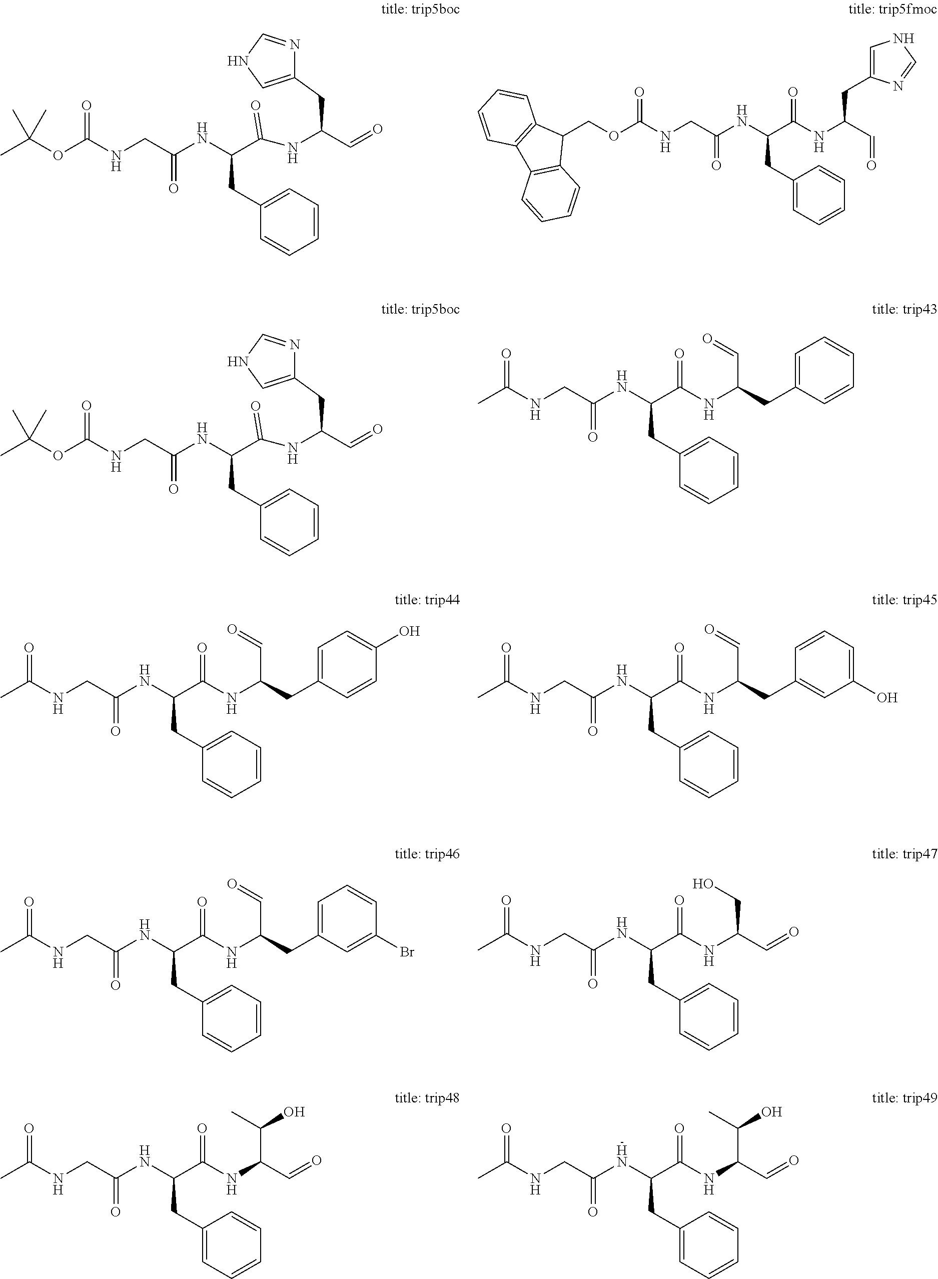

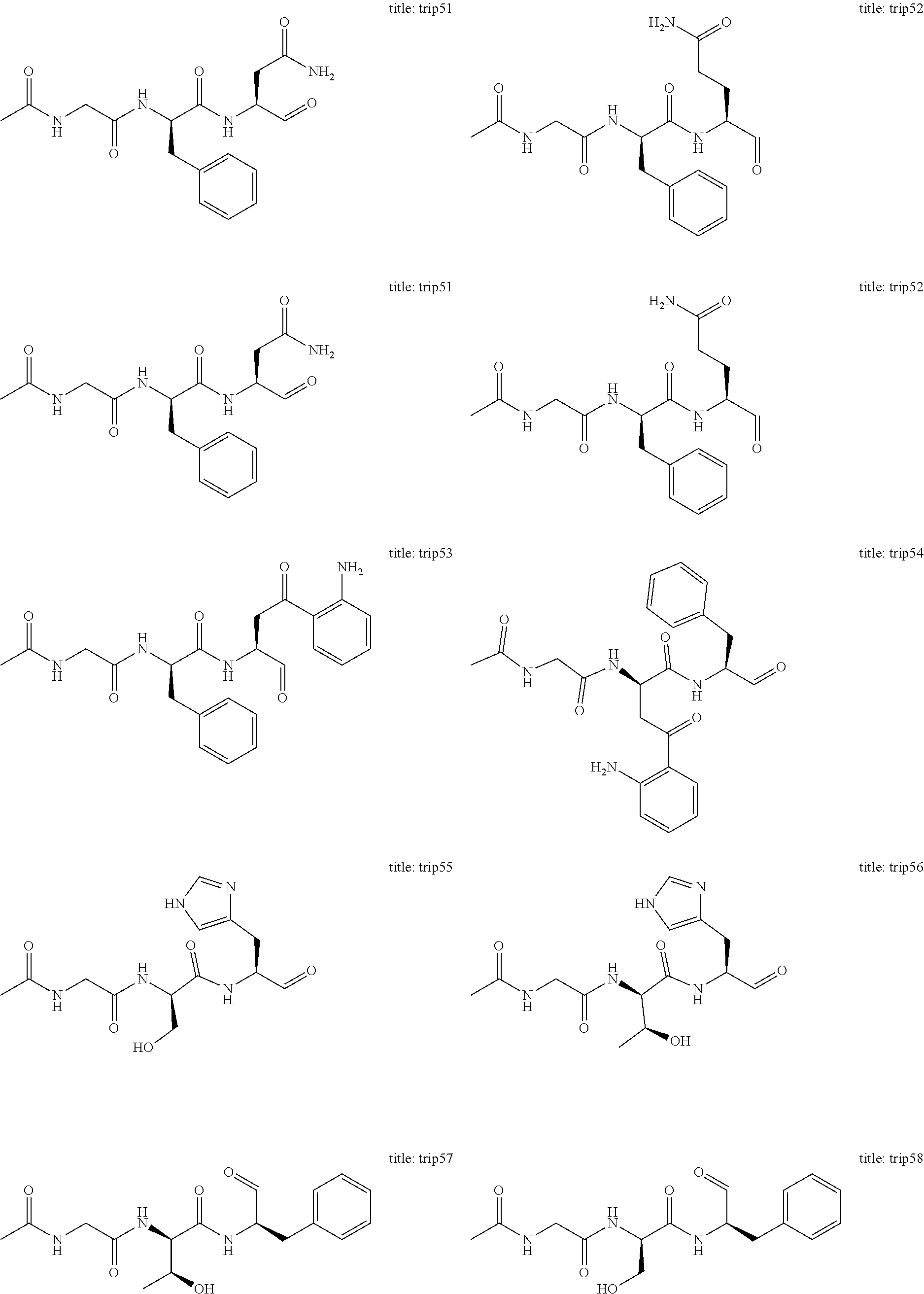

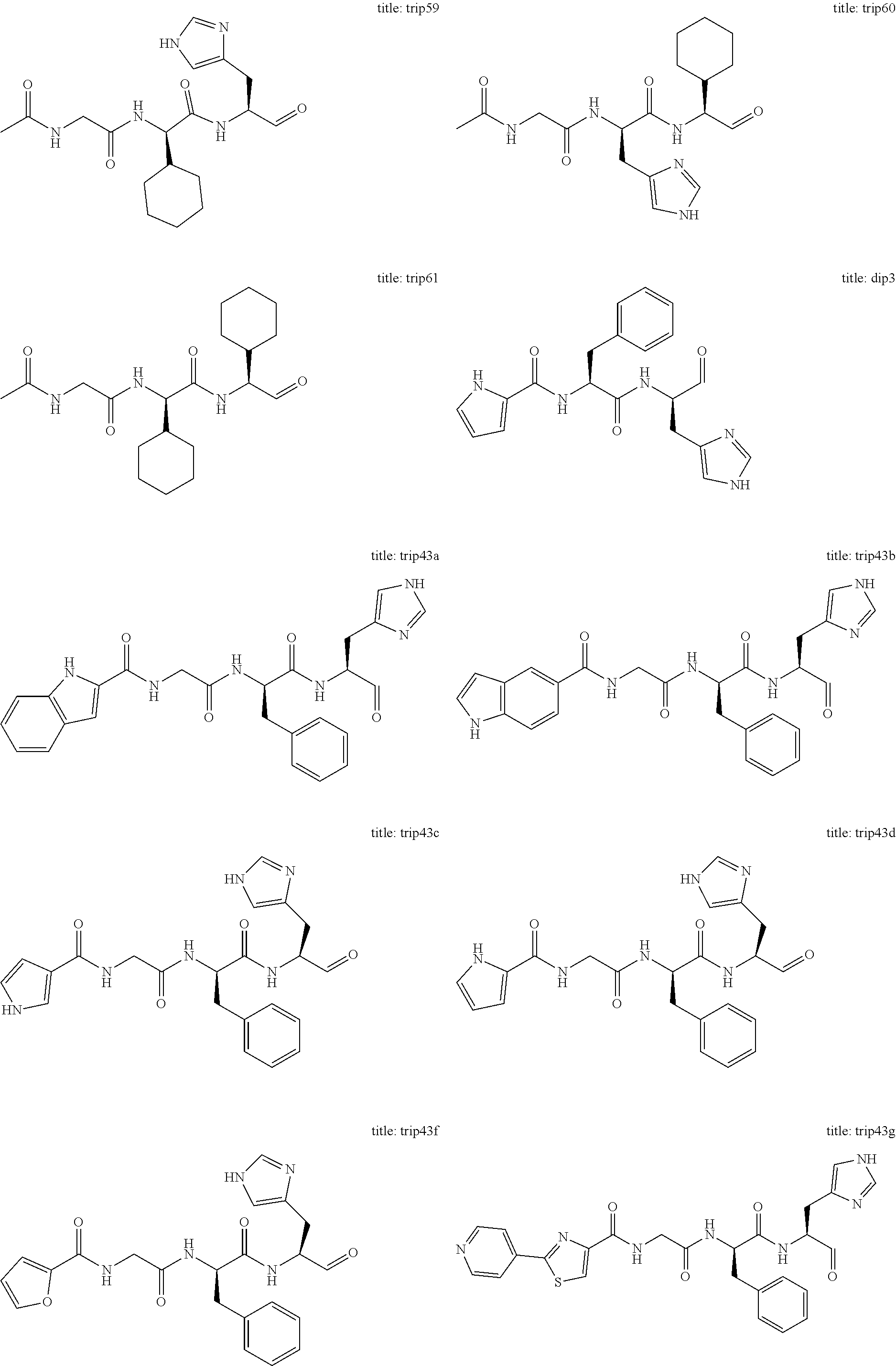

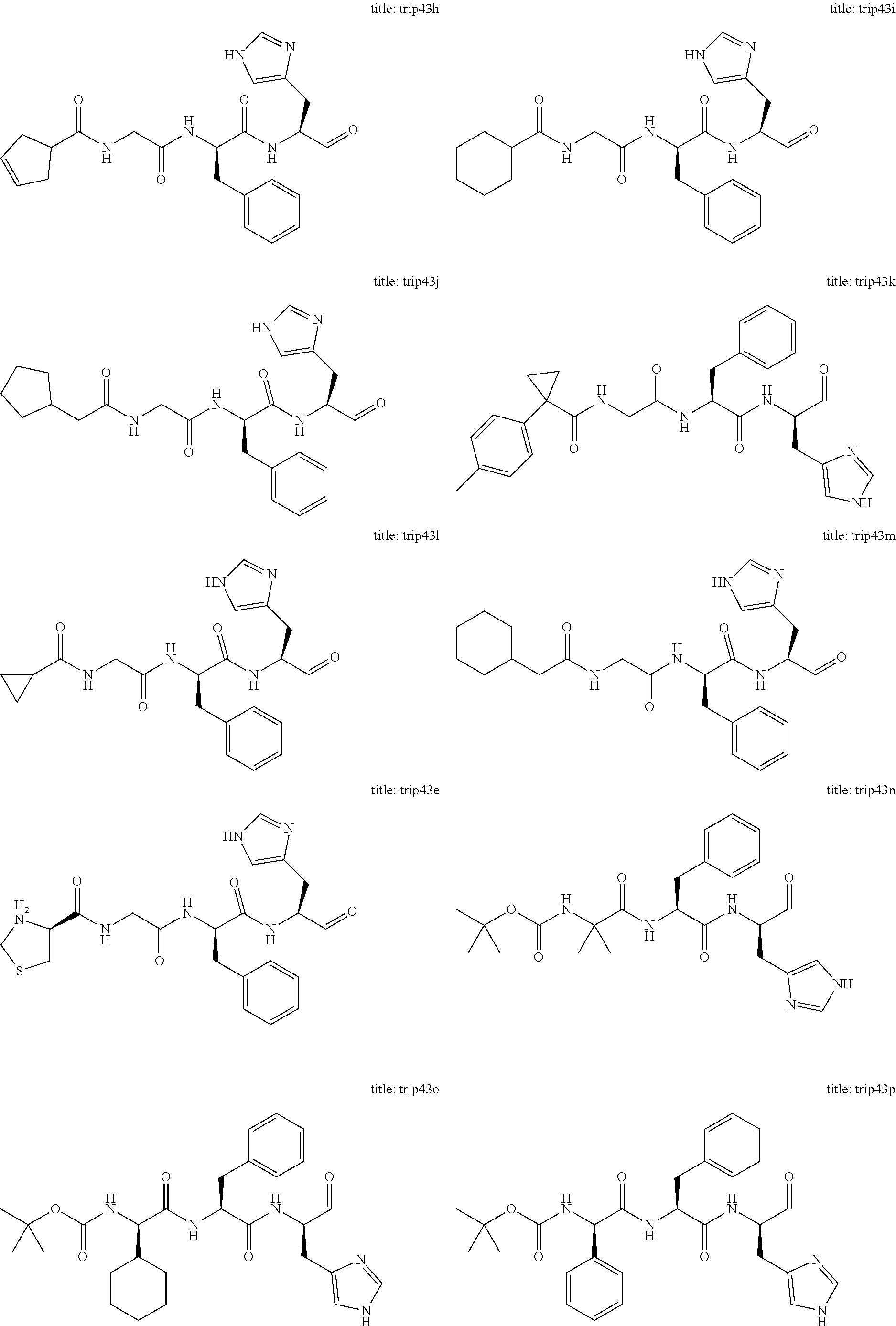

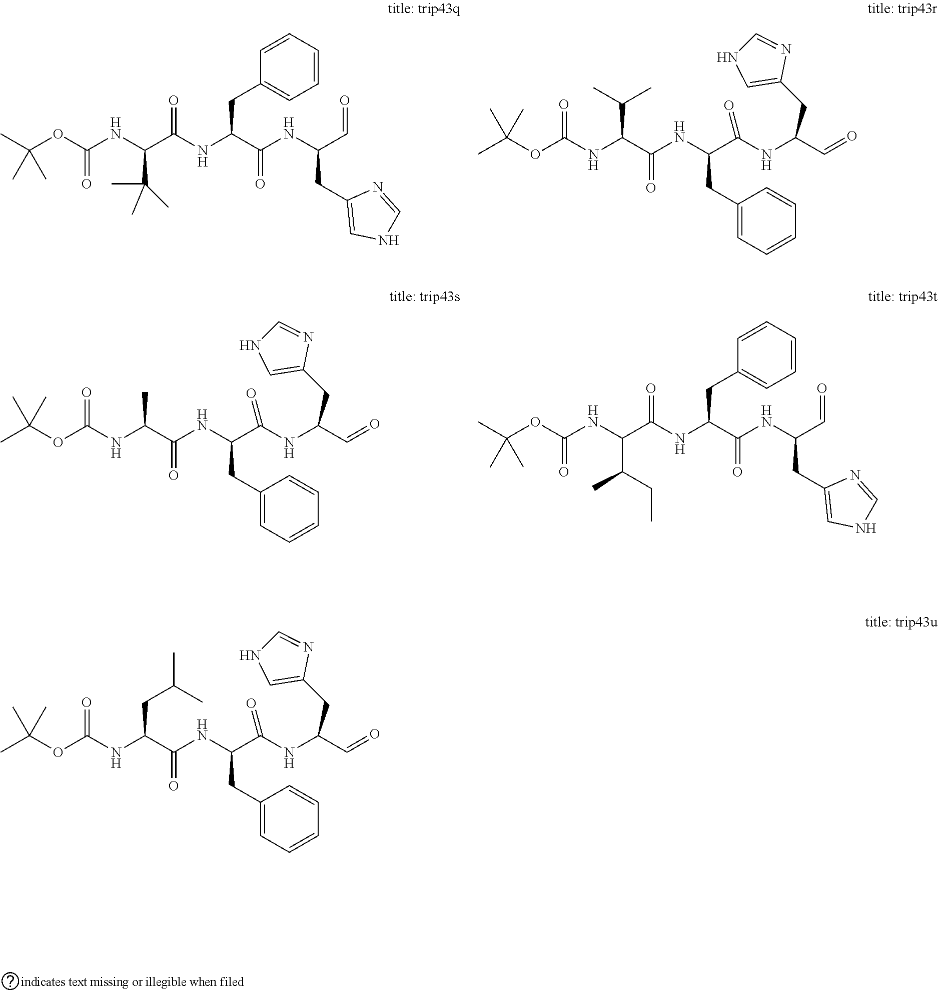 [SMILES: CC(C)(C)OC(=O)N[C@@H](C(=O)N[C@@H](Cc1ccccc1)C(=O)N[C@@H](C=O)Cc1c[nH]cn1)C(C)(C)C ; CC(C)[C@H](NC(=O)OC(C)(C)C)C(=O)N[C@H](Cc1ccccc1)C(=O)N[C@H](C=O)Cc1c[nH]cn1 ; C[C@H](NC(=O)OC(C)(C)C)C(=O)N[C@H](Cc1ccccc1)C(=O)N[C@H](C=O)Cc1c[nH]cn1 ; CC[C@@H](C)C(NC(=O)OC(C)(C)C)C(=O)N[C@@H](Cc1ccccc1)C(=O)N[C@@H](C=O)Cc1c[nH]cn1 ; CC(C)C[C@H](NC(=O)OC(C)(C)C)C(=O)N[C@H](Cc1ccccc1)C(=O)N[C@H](C=O)Cc1c[nH]cn1]

8. The peptide aldehyde according to claim 1, wherein said peptide is selected from the following:

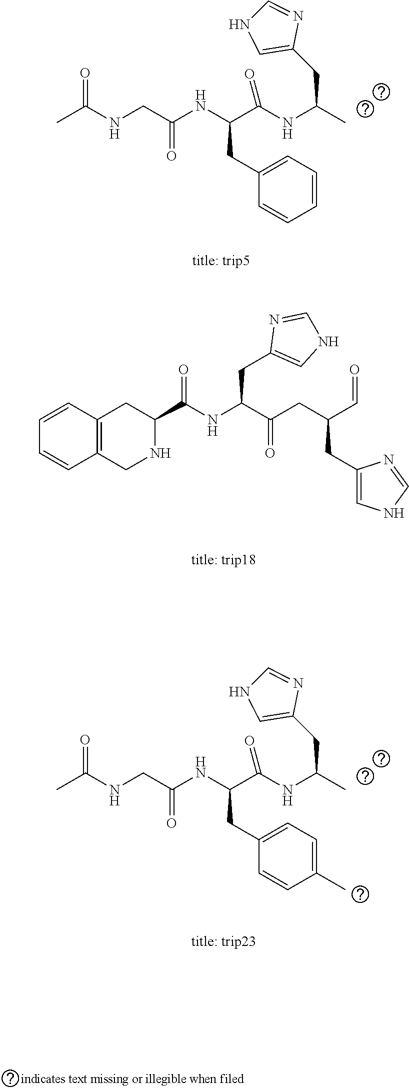

9. A pharmaceutical composition comprising at least one peptide, or salt or stereoisomer thereof, according to claim 1, and at least one pharmaceutically acceptable carrier and/or excipient.

10. The pharmaceutical composition according to claim 9, wherein said peptide, or salt or stereoisomer thereof is present in an amount ranging from 0.005 mg to 100 mg.

11. The pharmaceutical composition according to claim 9, in a form selected from solution, nasal drops, nasal aerosol and/or spray, nebulized solution or suspension, powder, microspheres, pill, capsule, tablet or beads.

12. The peptide or salt or stereoisomer thereof according to claim 1 or a pharmaceutical composition according to claim 9, for use in prevention and/or treatment of viral infections and/or of one or more diseases associated to or caused by viral infections.

13. The peptide or salt or stereoisomer thereof or the pharmaceutical composition for use according to claim 12, wherein said viral infection is a viral infection of the respiratory tract.

14. The peptide, or salt or stereoisomer thereof or the pharmaceutical composition for use according to claim 12, wherein said viral infection is a SARS-COV-2 infection.

15. The peptide, or salt or stereoisomer thereof or the pharmaceutical composition for use according to claim 12, wherein said one or more diseases associated to or caused by viral infections include interstitial pneumonia, acute lung injury (ALI), and acute respiratory distress syndrome (ARDS).

16. The peptide, or salt or stereoisomer thereof according to or the pharmaceutical composition for use according to claim 9, for use as inhibitor of the main protease (M^{pro}) of SARS-COV-2.

17. The peptide or salt or stereoisomer thereof or the pharmaceutical composition for use according to claim 12, wherein said compound or said pharmaceutical composition is administered to a subject in need thereof by way of inhalation, intra-nasal administration, nebulization, aerosol, or oral administration.

18. A nasal spray device comprising a peptide, or salt or stereoisomer thereof or a composition according to claim 1.

19. An oral composition comprising a peptide, or salt or stereoisomer thereof or a composition according to claim 1 in the form of gastroresistant microspheres.
